(19) United States
(12) Patent Application Publication
(10) Pub. No.: US 2007/0232813 A1
(43) Pub. Date: Oct. 4, 2007

(54) Substituted Cyclohexadienals - Syntheses and Applications

(76) Inventors: Coran M. H. Watanabe, College Station, TX (US); Bennie John Bench, College Station, TX (US)

Correspondence Address: FULBRIGHT & JAWORSKI L.L.P., 600 CONGRESS AVE., SUITE 2400, AUSTIN, TX, 78701, US

(21) Appl. No.: 11/692,780

(22) Filed: Mar. 28, 2007

Related U.S. Application Data
(60) Provisional application No. 60/786,542, filed on Mar. 28, 2006.

(51) Int. Cl. C07D 333/24; C07D 307/02; C07C 45/90

(52) U.S. Cl. 549/79; 568/446; 549/498; 568/433; 564/428

(57) ABSTRACT

The present invention is generally directed to the use of L-proline and certain derivatives thereof to catalyze the asymmetric self-condensation of α,β-unsaturated aldehydes to form homodimer and heterodimer cyclohexadienals. Reaction conditions are mild and yet amenable to a variety of different substrates yielding molecules with complex scaffolds from simple precursors. This approach allows for diversification and synthesis of this structural class of compounds in sufficient quantity, purity and enantioselectivity for, e.g., biological investigations and use as fluorescent probes, anti-cancer agents, anti-bacterial agents, and/or anti-fungal agents. The present invention is also generally directed to the cyclohexadienals produced.

This application claims the benefit of U.S. Provisional Application Ser. No. 60/786,542, filed Mar. 28, 2006, the entire contents of which are hereby incorporated by reference.

### BACKGROUND OF THE INVENTION

1. Field of the Invention

The present invention relates generally to the field of organic synthesis. More particularly, it concerns substituted cyclohexadiene aldehydes (cyclohexadienals) and their preparation using proline derivatives that are relatively inexpensive and readily accessible. As such, the present invention encompasses synthetic methods that involves a condensation reaction to produce the target cyclohexadienals as ring-fused homodimers and heterodimers in the presence of said proline derivatives. In certain embodiments, such methods allow preparation of a variety of cyclohexadienal-containing compounds, such as natural products, in sufficient quantity, purity and enantioselectivity for biological investigations. In certain embodiments, cyclohexadienals of the present invention will find use as fluorescent probes, anti-cancer agents, anti-bacterial agents, and/or anti-fungal agents.

2. Description of Related Art

Carotenoids form an important class of biologically active molecules playing a critical role in energy transfer processes such as photosynthesis or photoprotection. In animals, carotenoid metabolites e.g., retinoids, serve as chromophores for the visual signal transduction systems (Krinsky 1994).

Interestingly, condensation of all-E-retinal gives a C-40 ring-fused dimer A shown in FIG. 1 (with a cyclohexadienal structural core) that has been implicated as a contributor of age-related macular degeneration, the leading cause of blindness in the elderly (Fishkin et al., 2005; Fishkin et al., 2004). While molecules of this structural scaffold are not unprecedented in nature, considerably less is known regarding the biological functions of these molecules. The self-dimerization of citral, for example, has been suspected since the late 1890's (Tiemann, 1898; Labbé, 1899). The 1,2,4-trisubstituted structure B (shown in FIG. 1) was definitively ascribed in 1932 (Fischer and Löwenberg, 1932). Recently, citral dimer B was isolated from the North Sea bryozoan Flustra foliacea and shown to exhibit anti-bacterial activity against Roseobacter sp. and Sulfitobacter sp. at 100 μg resulting in inhibition of 0.5 and 1.0 cm, respectively, via agar diffusion assay (Holst et al., 1994; Peters et al., 2003; Peters et al., 2004).

Over the past century, a variety of conditions have been employed to develop facile routes to these self-condensation products. The general synthetic strategy has involved use of strongly basic conditions such as lithium diisopropyl amide (LDA) (Tucker et al., 1981), sodium hydride (NaH) (Verdegem et al., 1997; Taneja et al., 1988), potassium hydride (KH) (Tiemann, 1898; Labbé, 1899; Fischer and Löwenberg, 1932), potassium t-butoxide (Thomas and Guntz-Dubin, 1976), and others. The conditions utilized in these reactions, however, are fairly harsh and illustrate no degree of stereoselectivity. These aspects are commercially unattractive, not only in terms of costs of reagents and handling considerations, but products with little stereoselectivity are often not useful in biological testing settings. In certain situations, for example, one enantiomer has decidedly different biological properties than another enantiomer. Other reagents have been shown to catalyze the reaction of self-condensations, including trans-4-hydroxy-L-proline, D-proline and cis-4-hydroxy-D-proline (Asato et al., 1992). However, these reagents are relatively expensive and difficult to prepare (e.g., $5.00/g for trans-4-hydroxy-L-proline, $21/g for D-proline and $50.50/g for cis-4-hydroxy-D-proline, Sigma-Aldrich Co., Milwaukee, Wis.).

In view of the foregoing, it would be useful to have a process for synthesizing target cyclohexadienes that employs a mild, readily available and economical reagent.

### SUMMARY OF THE INVENTION

The present invention provides for novel cyclohexadienals and preparations thereof using a relatively mild, readily available and inexpensive reagent in comparison to other known reagents: L-proline and derivatives thereof. L-proline (roughly $0.50/g, Sigma-Aldrich Co., Milwaukee, Wis.) and various derivatives thereof have been discovered to effect an efficient synthesis of homodimers and heterodimers comprising cyclohexadienals. The methods discussed herein, in certain embodiments, allow for the preparation of these compounds in sufficient quantity, purity and enantioselectivity for, e.g., biological evaluation or use as fluorescent probes, anti-cancer agents, anti-bacterial agents, and/or anti-fungal agents.

Accordingly, the present invention contemplates a method of preparing a cyclohexadienal comprising reacting an α,β-unsaturated aldehyde with a β-substituted α,β-unsaturated aldehyde in the presence of a proline derivative. The proline derivative may be any such derivative known to those of skill in the art. In certain embodiments, the proline derivative is not trans-4-hydroxy-L-proline, D-proline or cis-4-hydroxy-D-proline. In certain embodiments, the proline derivative is a compound of formula (I):

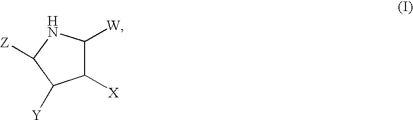

wherein W, X, Y, and Z are each independently selected from the group consisting of:
H;
halogen;
—COOR_{A}, wherein R_{A} is selected from the group consisting of H, alkyl, cycloalkyl and aryl;
—CR_{B}R_{C}R_{D}, wherein R_{B}, R_{C} and R_{D} are each independently selected from the group consisting of H, alkyl, cycloalkyl, aryl, —CN and halogen,
   or wherein R_{B}, R_{C}, and/or R_{D} taken together are comprised in a cyclic moiety with C,
   or wherein R_{C} is not present;
—C═N—R_{E}, wherein R_{E} is selected from the group consisting of alkyl, cycloalkyl and aryl;
—NR_{F}R_{G}, wherein R_{F} and R_{G} are independently selected from the group consisting of H, alkyl, cycloalkyl and aryl,
   or wherein R_{F} and R_{G} taken together are comprised in a cyclic moiety with N;
—N═R_{H}, wherein R_{H} is selected from the group consisting of alkyl, cycloalkyl and aryl; and
—OR_{I}, wherein R_{I} is selected from the group consisting of alkyl, cycloalkyl and aryl;
or wherein W and X, X and Y, and/or Y and Z taken together are comprised in a cyclic moiety;
   provided that the compound of formula (I) is not D-proline.

The proline derivative may catalyze the formation of a cyclohexadienal from an α,β-unsaturated aldehyde and a β-substituted α,β-unsaturated aldehyde. The α,β-unsaturated aldehyde and β-substituted α,β-unsaturated aldehyde may each be any known to those of skill in the art. In certain embodiments, the α,β-unsaturated aldehyde is a compound of formula (II):

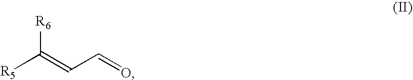

wherein R₅ and R₆ are each independently selected from the group consisting of:
H;
halogen;
—CR_{A}R_{B}R_{C}, wherein R_{A}, R_{B} and R_{C} are each independently selected from the group consisting of H, alkyl, cycloalkyl, aryl, —CN and halogen,
   or wherein R_{A}, R_{B}, and/or R_{C} taken together are comprised in a cyclic moiety with C,
   or wherein R_{C} is not present;
—C═N—R_{D}, wherein R_{D} is selected from the group consisting of alkyl, cycloalkyl and aryl;
—NR_{E}R_{F}, wherein R_{E} and R_{F} are independently selected from the group consisting of H, alkyl, cycloalkyl and aryl,
   or wherein R_{E} and R_{F} taken together are comprised in a cyclic moiety with N;
—N═R_{G}, wherein R_{G} is selected from the group consisting of alkyl, cycloalkyl and aryl; and
—OR_{H}, wherein R_{H} is selected from the group consisting of alkyl, cycloalkyl and aryl;
or wherein R₅ and R₆ taken together are comprised in a cyclic moiety.

In certain embodiments, the β-substituted α,β-unsaturated aldehyde is a compound of formula (III):

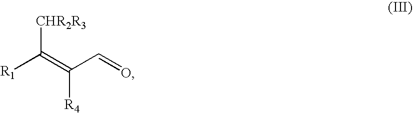

wherein R₁, R₂, R₃, and R₄ are each independently selected from the group consisting of:
—H;
halogen;
—CR_{A}R_{B}R_{C}, wherein R_{A}, R_{B} and R_{C} are each independently selected from the group consisting of H, alkyl, cycloalkyl, aryl, —CN and halogen,
   or wherein R_{A}, R_{B}, and/or R_{C} taken together are comprised in a cyclic moiety with C
   or wherein R_{C} is not present;
—C═N—R_{D}, wherein R_{D} is selected from the group consisting of alkyl, cycloalkyl and aryl;
—NR_{E}R_{F}, wherein R_{E} and R_{F} are independently selected from the group consisting of H, alkyl, cycloalkyl and aryl,
   or wherein R_{E} and R_{F} taken together are comprised in a cyclic moiety with N;
—N═R_{G}, wherein R_{G} is selected from the group consisting of alkyl, cycloalkyl and aryl; and
—OR_{H}, wherein R_{H} is selected from the group consisting of alkyl, cycloalkyl and aryl,
or wherein R₁ and R₂, R₁ and R₄, and/or R₂ and R₃ taken together are comprised in a cyclic moiety.

In certain methods of the present invention, W of the compound of formula (I) is COOR_{A}. R_{A} may be H, in certain embodiments. In certain embodiments, R_{A} is an alkyl group, such as a methyl, isopropyl, or benzyl group. W may also be, in certain embodiments, an alkyl group, such as an alkyl group selected from the group consisting of CH₂OH, —C(H)(OH)((CH₂)(CH₃))₂ and —C(H)(OH)(phenyl)₂. In certain embodiments, X, Y and Z are each H. In certain embodiments, the compound of formula (I) is further defined as L-proline. In certain embodiments, the α,β-unsaturated aldehyde is the same compound as the β-substituted α,β-unsaturated aldehyde. In such situations, the molar ratio of the aldehyde to proline derivative may be about, at least about, or at most about 1:0.25, 1:0.5, 1:0.75, 1:1, 1:1.25, 1:1.5, 1:1.75, 1:2, 1:2.5 or 1:3. In certain embodiments, the ratio is about 1:1.5. In certain embodiments, the α,β-unsaturated aldehyde is a different compound than the β-substituted α,β-unsaturated aldehyde.

Between the compounds of formula (II) and (III), R₁—R₆ may each individually be any substituent known to those of skill in the art. For example, in certain embodiments, R₁, R₂, R₃, R₄, R₅ and R₆ may each independently be selected from the group consisting of H, alkyl, cycloalkyl and aryl. In certain embodiments, only one of R₁, R₂, R₃, R₄, R₅ and R₆ is H. In certain embodiments, only one of R₂ and R₃ is H. In certain embodiments, cyclohexadienals of the present invention are substantially pure, as defined herein. In certain embodiments, the substantially pure cyclohexadienal is between 10-62% ee. Cyclohexadienals produced via the method of the present invention may be acquired in yields ranging from about, or at least about 5%, 10%, 15%, 20%, 25%, 30%, 35%, 40%, 45%, 50%, 55%, 60%, 65%, 70%, 75%, 80%, 85%, 90%, 95%, or 99%, or any range derivable therein.

Cyclohexadienals of the present invention may naturally spontaneously aromatize or may aromatize in the presence of external stimulation, such as light, heat, and/or additional reagents. The products of said aromatization may be fluorescent. Typically, the fluorescent product contains conjugated double bonds. In non-limiting examples, cyclohexadienals that aromatize to produce fluorescent products may comprise a naphthyl, phenyl, or farnesyl group attached, directly or indirectly, to the cyclohexadienal. In certain embodiments, a fluorescent product is produced via any method described herein when when R₅ or R₆ of a cyclohexadienal of the present invention (stemming from a compound of formula (III)) is an aryl group. The aryl group may be a phenyl group. In certain embodiments, a cyclohexadienyl that spontaneously aromatizes may be further defined as 4-(4-methyl-pent-3-enyl)-6-(4-nitro-phenyl)-cyclohexa-1,3-dienecarbaldehyde. In certain embodiments, the present invention contemplates a method of preparing a fluorescent compound, comprising reacting a cyclohexadienal with potassium permanganate under acidic conditions, as described herein. A cyclohexadienal that may aromatize under these conditions may be defined as, in certain embodiments, 4-(4-methylpent-3-enyl)-6-phenylcyclohexa-1,3-dienecarbaldehyde.

As discussed, a cyclohexadienal produced by the methods described herein may be any cyclohexadienal known to those of skill in the art. The cyclohexadienal may also be a cyclohexadienal that is presently unknown, but discovered at a later date. A cyclohexadienal of the present invention may be further comprised in a pharmaceutical composition. In certain embodiments, a cyclohexadiene produced by the methods described herein is selected from the group consisting of 6-methyl-4,6-bis-(4-methyl-pent-3-enyl)-cyclohexa-1,3-dienecarbaldehyde; 6,6-dimethyl-4-(4-methyl-pent-3-enyl)-cyclohexa-1,3-dienecarbaldehyde; 6-methyl-4-(4-methyl-pent-3-enyl)-cyclohexa-1,3-dienecarbaldehyde; 4-(4-methyl-pent-3-enyl)-6-phenyl-cyclohexa-1,3-dienecarbaldehyde; 4-(4-methyl-pent-3-enyl)-6-(4-nitro-phenyl)-cyclohexa-1,3-dienecarbaldehyde; 6-(4-dimethylamino-phenyl)-4-(4-methyl-pent-3-enyl)-cyclohexa-1,3-dienecarbaldehyde; 4-(4-methyl-pent-3-enyl)-cyclohexa-1,3-dienecarbaldehyde; 6-methyl-4,6-bis-(5-methyl-furan-2-yl)-cyclohexa-1,3-dienecarbaldehyde; 4-(5-methyl-furan-2-yl)-6-phenyl-cyclohexa-1,3-dienecarbaldehyde; 4-(5-methyl-furan-2-yl)-6-(4-nitro-phenyl)-cyclohexa-1,3-dienecarbaldehyde; 6-(4-dimethylamino-phenyl)-4-(5-methyl-furan-2-yl)-cyclohexa-1,3-dienecarbaldehyde; 6-methyl-4,6-di-thiophen-2-yl-cyclohexa-1,3-dienecarbaldehyde; 6-(4-dimethylamino-phenyl)-4-thiophen-2-yl-cyclohexa-1,3-dienecarbaldehyde; 6-(4-nitro-phenyl)-4-thiophen-2-yl-cyclohexa-1,3-dienecarbaldehyde; 6-phenyl-4-thiophen-2-yl-cyclohexa-1,3-dienecarbaldehyde; 6-methyl-4-thiophen-2-yl-cyclohexa-1,3-dienecarbaldehyde; 6-(4-dimethylamino-phenyl)-4-(1H-indol-3-yl)-cyclohexa-1,3-dienecarbaldehyde; 4,6-bis-(1H-indol-3-yl)-6-methyl-cyclohexa-1,3-dienecarbaldehyde; 4-methyl-6-phenyl-cyclohexa-1,3-dienecarbaldehyde; 4-hept-1-enyl-6-methyl-cyclohexa-1,3-dienecarbaldehyde; 4,6-di-hept-1-enyl-6-methyl-cyclohexa-1,3-dienecarbaldehyde; 4,6-bis-biphenyl-4-yl-6-methyl-cyclohexa-1,3-dienecarbaldehyde; 4-biphenyl-4-yl-6-methyl-cyclohexa-1,3-dienecarbaldehyde; 4-biphenyl-4-yl-6-phenyl-cyclohexa-1,3-dienecarbaldehyde; 4-biphenyl-4-yl-6-(4-nitro-phenyl)-cyclohexa-1,3-dienecarbaldehyde; 4-biphenyl-4-yl-6-(4-dimethylamino-phenyl)-cyclohexa-1,3-dienecarbaldehyde; 4,6-bis-(9H-fluoren-2-yl)-6-methyl-cyclohexa-1,3-dienecarbaldehyde; 4-(9H-fluoren-2-yl)-6-methyl-cyclohexa-1,3-dienecarbaldehyde; 4-(9H-fluoren-2-yl)-6-phenyl-cyclohexa-1,3-dienecarbaldehyde; 6-(4-dimethylamino-phenyl)-4-(9H-fluoren-2-yl)-cyclohexa-1,3-dienecarbaldehyde; 4-(9H-fluoren-2-yl)-6-(4-nitro-phenyl)-cyclohexa-1,3-dienecarbaldehyde; 6-methyl-4-[2-(2,6,6-trimethyl-cyclohex-1-enyl)-vinyl]-cyclohexa-1,3-dienecarbaldehyde; 6-phenyl-4-[2-(2,6,6-trimethyl-cyclohex-1-enyl)-vinyl]-cyclohexa-1,3-dienecarbaldehyde; 6-(4-nitro-phenyl)-4-[2-(2,6,6-trimethyl-cyclohex-1-enyl)-vinyl]-cyclohexa-1,3-dienecarbaldehyde; 6-(4-dimethylamino-phenyl)-4-[2-(2,6,6-trimethyl-cyclohex-1-enyl)-vinyl]-cyclohexa-1,3-dienecarbaldehyde; 6-methyl-4,6-bis-[2-(2,6,6-trimethyl-cyclohex-1-enyl)-vinyl]-cyclohexa-1,3-dienecarbaldehyde; 6-methyl-6-[3-methyl-6-(2,6,6-trimethyl-cyclohex-1-enyl)-hexa-1,3,5-trienyl]-4-[4-methyl-6-(2,6,6-trimethyl-cyclohex-1-enyl)-hexa-1,3,5-trienyl]-cyclohexa-1,3-dienecarbaldehyde; 6-methyl-4-[4-methyl-6-(2,6,6-trimethyl-cyclohex-1-enyl)-hexa-1,3,5-trienyl]-cyclohexa-1,3-dienecarbaldehyde; 4-[4-methyl-6-(2,6,6-trimethyl-cyclohex-1-enyl)-hexa-1,3,5-trienyl]-6-phenyl-cyclohexa-1,3-dienecarbaldehyde; 6-(4-dimethylamino-phenyl)-4-[4-methyl-6-(2,6,6-trimethyl-cyclohex-1-enyl)-hexa-1,3,5-trienyl]-cyclohexa-1,3-dienecarbaldehyde; 4-[4-methyl-6-(2,6,6-trimethyl-cyclohex-1-enyl)-hexa-1,3,5-trienyl]-6-(4-nitro-phenyl)-cyclohexa-1,3-dienecarbaldehyde; 6-methyl-4-(4-methyl-pent-3-enyl)-6-thiophen-2-yl-cyclohexa-1,3-dienecarbaldehyde; 4-biphenyl-4-yl-6-methyl-6-(5-methyl-furan-2-yl)-cyclohexa-1,3-dienecarbaldehyde; 4,6-dihexyl-6-methyl-cyclohexa-1,3-dienecarbaldehyde; 4-hexyl-6-methyl-cyclohexa-1,3-dienecarbaldehyde; 4-biphenyl-4-yl-6-methyl-6-thiophen-2-yl-cyclohexa-1,3-dienecarbaldehyde; (E)-6,6-dimethyl-4-(2-(2,6,6-trimethylcyclohex-1-enyl)vinyl)cyclohexa-1,3-dienecarbaldehyde; 6-methyl-6-(5-methylfuran-2-yl)-4-(4-methylpent-3-enyl)cyclohexa-1,3-dienecarbaldehyde; 6,6-dimethyl-4-(5-methylfuran-2-yl)cyclohexa-1,3-dienecarbaldehyde; 6-methyl-4-(naphthalen-2-yl)cyclohexa-1,3-dienecarbaldehyde; (E)-4-(4,8-dimethylnona-3,7-dienyl)-6-methylcyclohexa-1,3-dienecarbaldehyde; 5,6-dimethyl-4-(thiophen-2-yl)cyclohexa-1,3-dienecarbaldehyde; 5,6,6-trimethyl-4-(thiophen-2-yl)cyclohexa-1,3-dienecarbaldehyde; 6-(biphenyl-4-yl)-4,6-dimethylcyclohexa-1,3-dienecarbaldehyde; 6,6-dimethyl-4-(naphthalen-2-yl)cyclohexa-1,3-dienecarbaldehyde; 4-(biphenyl-4-yl)-6,6-dimethylcyclohexa-1,3-dienecarbaldehyde; 4,6,6-trimethylcyclohexa-1,3-dienecarbaldehyde; 5,6-dimethyl-4,6-di(thiophen-2-yl)cyclohexa-1,3-dienecarbaldehyde; 6-methyl-4,6-diphenylcyclohexa-1,3-dienecarbaldehyde; 6-methyl-4,6-di(naphthalen-2-yl)cyclohexa-1,3-dienecarbaldehyde; 4,6-bis((E)-4,8-dimethylnona-3,7-dienyl)-6-methylcyclohexa-1,3-dienecarbaldehyde; cis-4-methyl-5-(3-methyl-but-2-enyl)-6-phenyl-cyclohexa-1,3-dienecarbaldehyde; trans-4-methyl-5-(3-methyl-but-2-enyl)-6-phenyl-cyclohexa-1,3-dienecarbaldehyde; cis and trans-6-(4-dimethylamino-phenyl)-4-methyl-5-(3-methyl-but-2-enyl)-cyclohexa-1,3-dienecarbaldehyde; 6-(4-(dimethylamino)phenyl)-4-methylcyclohexa-1,3-dienecarbaldehyde; 4-methyl-6-(4-nitrophenyl)cyclohexa-1,3-dienecarbaldehyde; 4-methylcyclohexa-1,3-dienecarbaldehyde; 4-hexyl-6-phenylcyclohexa-1,3-dienecarbaldehyde; 6-(4-(dimethylamino)phenyl)-4-hexylcyclohexa-1,3-dienecarbaldehyde; 4,6-diphenylcyclohexa-1,3-dienecarbaldehyde; 6-(4-(dimethylamino)phenyl)-4-phenylcyclohexa-1,3-dienecarbaldehyde; 6-(4-nitrophenyl)-4-phenylcyclohexa-1,3-dienecarbaldehyde; 4-(naphthalen-2-yl)-6-phenylcyclohexa-1,3-dienecarbaldehyde; 6-(4-(dimethylamino)phenyl)-4-(naphthalen-2-yl)cyclohexa-1,3-dienecarbaldehyde; 4-(naphthalen-2-yl)-6-(4-nitrophenyl)cyclohexa-1,3-dienecarbaldehyde; 4-(naphthalen-2-yl)cyclohexa-1,3-dienecarbaldehyde; (E)-4-(4,8-dimethylnona-3,7-dienyl)cyclohexa-1,3-dienecarbaldehyde; (E)-4-(4,8-dimethylnona-3,7-dienyl)-6-phenylcyclohexa-1,3-dienecarbaldehyde; (E)-6-(4-(dimethylamino)phenyl)-4-(4,8-dimethylnona-3,7-dienyl)cyclohexa-1,3-dienecarbaldehyde; 5-methyl-6-(4-nitrophenyl)-4-(thiophen-2-yl)cyclohexa-1,3-dienecarbaldehyde; 6-(4-(dimethylamino)phenyl)-5-methyl-4-(thiophen-2-yl)cyclohexa-1,3-dienecarbaldehyde; 5-methyl-6-phenyl-4-(thiophen-2-yl)cyclohexa-1,3-dienecarbaldehyde; 5-methyl-4-(thiophen-2-yl)cyclohexa-1,3-dienecarbaldehyde; 5-(4-methyl-pent-3-enyl)-4′-nitro-biphenyl-2-carbaldehyde; 4″-dimethylamino-[1,1′;3′,1″]terphenyl-4′-carbaldehyde; 4′″-dimethylamino-[1,1′;4′,1″;3″,1′″]quaterphenyl-4″-carbaldehyde; 4′-dimethylamino-5-(9H-fluoren-2-yl)-biphenyl-2-carbaldehyde; 4′-(dimethylamino)-5-methylbiphenyl-2-carbaldehyde; 5-(9H-fluoren-2-yl)-biphenyl-2-carbaldehyde; and 5-(4-methyl-pent-3-enyl)-biphenyl-2-carbaldehyde.

The synthetic methods of the present invention may take place in any solvent known to those of skill in the art that does not impede the desired reaction. Solvent choices for the methods of the present invention will be known to one of ordinary skill in the art. Solvent choices may depend, for example, on which one(s) will facilitate the solubilizing of all the reagents or, for example, which one(s) will best facilitate the desired reaction (particularly when the mechanism of the reaction is known). Solvents may include, for example, polar solvents or non-polar solvents. Solvents choices include, but are not limited to, tetrahydrofuran, dimethylformamide, dimethylsulfoxide, dioxane, methanol, ethanol, hexane, methylene chloride and acetonitrile. In some embodiments, the solvent is ethanol. More than one solvent may be chosen for any particular reaction or purification procedure. Water may also be admixed into any solvent choice. Further, water, such as distilled water, may constitute the reaction medium instead of a solvent.

Methods of the present invention also contemplate methods of stimulating neurite growth, comprising administering to a cell an effective amount of a cyclohexadienal as described herein. In certain embodiments, the cyclohexadienal comprises a substituent selected from the group consisting of naphthyl, biphenyl and farnesyl. The cyclohexadienal that stimulates neurite growth may be selected from the group consisting of 4-(biphenyl-4-yl)-6-methyl-6-(5-methylfuran-2-yl)cyclohexa-1,3-dienecarbaldehyde, 4,6-dihexyl-6-methylcyclohexa-1,3-dienecarbaldehyde and 6-(4-(dimethylamino)phenyl)-4-(naphthalen-2-yl)cyclohexa-1,3-dienecarbaldehyde.

In certain embodiments, the present invention contemplates an improved method for reacting an α,β-unsaturated aldehyde with a β-substituted α,β-unsaturated aldehyde, the improvement comprising performing the reaction in the presence of a proline derivative. Such a proline derivative may be of the type known to those of skill in the art, but to the exclusion of trans-4-hydroxy-L-proline, D-proline and cis-4-hydroxy-D-proline. In certain embodiments, the proline derivative is a compound of formula (I):

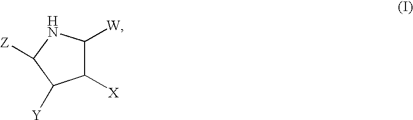

wherein W, X, Y, and Z are each independently selected from the group consisting of:
H;
halogen;
—COOR_{A}, wherein R_{A} is selected from the group consisting of H, alkyl, cycloalkyl and aryl;
—CR_{B}R_{C}R_{D}, wherein R_{B}, R_{C} and R_{D} are each independently selected from the group consisting of H, alkyl, cycloalkyl, aryl, —CN and halogen,
   or wherein R_{B}, R_{C}, and/or R_{D} taken together are comprised in a cyclic moiety with C,
   or wherein R_{C} is not present;
—C═N—R_{E}, wherein R_{E} is selected from the group consisting of alkyl, cycloalkyl and aryl;
—NR_{F}R_{G}, wherein R_{F} and R_{G} are independently selected from the group consisting of H, alkyl, cycloalkyl and aryl,
   or wherein R_{F} and R_{G} taken together are comprised in a cyclic moiety with N;
—N═R_{H}, wherein R_{H} is selected from the group consisting of alkyl, cycloalkyl and aryl; and
—OR_{I}, wherein R_{I} is selected from the group consisting of alkyl, cycloalkyl and aryl;
or wherein W and X, X and Y, and/or Y and Z taken together are comprised in a cyclic moiety;
   provided that the compound of formula (I) is not D-proline.

Persons of ordinary skill in the art will be familiar with methods of purifying compounds of the present invention, such as the cyclohexadienals as described herein. One of ordinary skill in the art will understand that compounds of the present invention can generally be purified at any step, including the purification of intermediates as well as purification of the final products. In certain embodiments, purification is performed via silica gel column chromatography or HPLC. In certain embodiments, a chiral column may be employed in chromatographic methods to obtain a desired enantiomer.

The present invention also contemplates cyclohexadienals. In certain embodiments, the present invention contemplates cyclohexadienals produced via synthetic methods mediated by a proline derivative, as described herein. In certain embodiments, the cyclohexadienal is a compound of formula (IV):

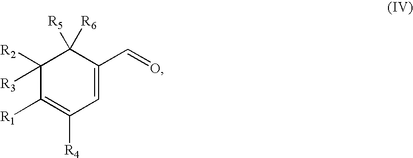

wherein R₁, R₂, R₃, R₄, R₅ and R₆ are each independently selected from the group consisting of:
H;
halogen;
—CR_{A}R_{B}R_{C}, wherein R_{A}, R₁B and R_{C} are each independently selected from the group consisting of H, alkyl, cycloalkyl, aryl, —CN and halogen,
   or wherein R_{A}, R_{B}, and/or R_{C} taken together are comprised in a cyclic moiety with C,
   or wherein R_{C} is not present;
—C═N—R_{D}, wherein R_{D} is selected from the group consisting of alkyl, cycloalkyl and aryl;
—NR_{E}R_{F}, wherein R_{E} and R_{F} are independently selected from the group consisting of H, alkyl, cycloalkyl and aryl,
   or wherein R_{E} and R_{F} taken together are comprised in a cyclic moiety with N;
—N═R_{G}, wherein R_{G} is selected from the group consisting of alkyl, cycloalkyl and aryl; and
—OR_{H}, wherein R_{H} is selected from the group consisting of alkyl, cycloalkyl and aryl;
or wherein R₁ and R₂, R₁ and R₄, R₂ and R₃, and/or R₅ and R₆ taken together form a cyclic moiety.

In certain embodiments, the compound of formula (IV) is neither A nor B:

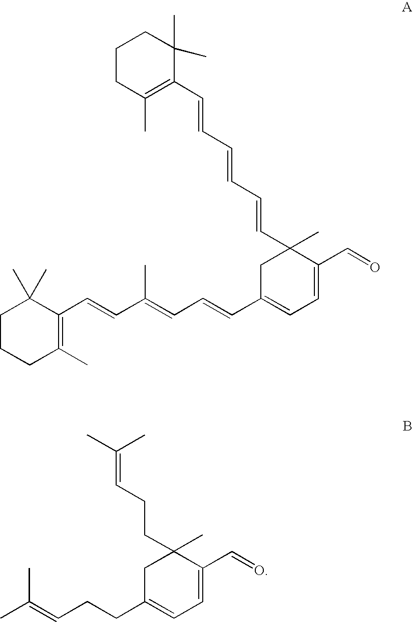

In certain embodiments, R₁ of the cyclohexadienal of formula (IV) is selected from the group consisting of alkyl, cycloalkyl and aryl. R₅ and R₆ of the cyclohexadienal of formula (IV) may each be independently selected from the group consisting of H, alkyl, cycloalkyl and aryl, in certain embodiments. In particular embodiments, R₅ or R₆ is an aryl group, such as a phenyl group. The cyclohexadienal of formula (IV) may be further defined, in certain embodiments, as 6-methyl-4,6-bis-(4-methyl-pent-3-enyl)-cyclohexa-1,3-dienecarbaldehyde; 6,6-dimethyl-4-(4-methyl-pent-3-enyl)-cyclohexa-1,3-dienecarbaldehyde; 6-methyl-4-(4-methyl-pent-3-enyl)-cyclohexa-1,3-dienecarbaldehyde; 4-(4-methyl-pent-3-enyl)-6-phenyl-cyclohexa-1,3-dienecarbaldehyde; 4-(4-methyl-pent-3-enyl)-6-(4-nitro-phenyl)-cyclohexa-1,3-dienecarbaldehyde; 6-(4-dimethylamino-phenyl)-4-(4-methyl-pent-3-enyl)-cyclohexa-1,3-dienecarbaldehyde; 4-(4-methyl-pent-3-enyl)-cyclohexa-1,3-dienecarbaldehyde; 6-methyl-4,6-bis-(5-methyl-furan-2-yl)-cyclohexa-1,3-dienecarbaldehyde; 4-(5-methyl-furan-2-yl)-6-phenyl-cyclohexa-1,3-dienecarbaldehyde; 4-(5-methyl-furan-2-yl)-6-(4-nitro-phenyl)-cyclohexa-1,3-dienecarbaldehyde; 6-(4-dimethylamino-phenyl)-4-(5-methyl-furan-2-yl)-cyclohexa-1,3-dienecarbaldehyde; 6-methyl-4,6-di-thiophen-2-yl-cyclohexa-1,3-dienecarbaldehyde; 6-(4-dimethylamino-phenyl)-4-thiophen-2-yl-cyclohexa-1,3-dienecarbaldehyde; 6-(4-nitro-phenyl)-4-thiophen-2-yl-cyclohexa-1,3-dienecarbaldehyde; 6-phenyl-4-thiophen-2-yl-cyclohexa-1,3-dienecarbaldehyde; 6-methyl-4-thiophen-2-yl-cyclohexa-1,3-dienecarbaldehyde; 6-(4-dimethylamino-phenyl)-4-(1H-indol-3-yl)-cyclohexa-1,3-dienecarbaldehyde; 4,6-bis-(1H-indol-3-yl)-6-methyl-cyclohexa-1,3-dienecarbaldehyde; 4-methyl-6-phenyl-cyclohexa-1,3-dienecarbaldehyde; 4-hept-1-enyl-6-methyl-cyclohexa-1,3-dienecarbaldehyde; 4,6-di-hept-1-enyl-6-methyl-cyclohexa-1,3-dienecarbaldehyde; 4,6-bis-biphenyl-4-yl-6-methyl-cyclohexa-1,3-dienecarbaldehyde; 4-biphenyl-4-yl-6-methyl-cyclohexa-1,3-dienecarbaldehyde; 4-biphenyl-4-yl-6-phenyl-cyclohexa-1,3-dienecarbaldehyde; 4-biphenyl-4-yl-6-(4-nitro-phenyl)-cyclohexa-1,3-dienecarbaldehyde; 4-biphenyl-4-yl-6-(4-dimethylamino-phenyl)-cyclohexa-1,3-dienecarbaldehyde; 4,6-bis-(9H-fluoren-2-yl)-6-methyl-cyclohexa-1,3-dienecarbaldehyde; 4-(9H-fluoren-2-yl)-6-methyl-cyclohexa-1,3-dienecarbaldehyde; 4-(9H-fluoren-2-yl)-6-phenyl-cyclohexa-1,3-dienecarbaldehyde; 6-(4-dimethylamino-phenyl)-4-(9H-fluoren-2-yl)-cyclohexa-1,3-dienecarbaldehyde; 4-(9H-fluoren-2-yl)-6-(4-nitro-phenyl)-cyclohexa-1,3-dienecarbaldehyde; 6-methyl-4-[2-(2,6,6-trimethyl-cyclohex-1-enyl)-vinyl]-cyclohexa-1,3-dienecarbaldehyde; 6-phenyl-4-[2-(2,6,6-trimethyl-cyclohex-1-enyl)-vinyl]-cyclohexa-1,3-dienecarbaldehyde; 6-(4-nitro-phenyl)-4-[2-(2,6,6-trimethyl-cyclohex-1-enyl)-vinyl]-cyclohexa-1,3-dienecarbaldehyde; 6-(4-dimethylamino-phenyl)-4-[2-(2,6,6-trimethyl-cyclohex-1-enyl)-vinyl]-cyclohexa-1,3-dienecarbaldehyde; 6-methyl-4,6-bis-[2-(2,6,6-trimethyl-cyclohex-1-enyl)-vinyl]-cyclohexa-1,3-dienecarbaldehyde; 6-methyl-6-[3-methyl-6-(2,6,6-trimethyl-cyclohex-1-enyl)-hexa-1,3,5-trienyl]-4-[4-methyl-6-(2,6,6-trimethyl-cyclohex-1-enyl)-hexa-1,3,5-trienyl]-cyclohexa-1,3-dienecarbaldehyde; 6-methyl-4-[4-methyl-6-(2,6,6-trimethyl-cyclohex-1-enyl)-hexa-1,3,5-trienyl]-cyclohexa-1,3-dienecarbaldehyde; 4-[4-methyl-6-(2,6,6-trimethyl-cyclohex-1-enyl)-hexa-1,3,5-trienyl]-6-phenyl-cyclohexa-1,3-dienecarbaldehyde; 6-(4-dimethylamino-phenyl)-4-[4-methyl-6-(2,6,6-trimethyl-cyclohex-1-enyl)-hexa-1,3,5-trienyl]-cyclohexa-1,3-dienecarbaldehyde; 4-[4-methyl-6-(2,6,6-trimethyl-cyclohex-1-enyl)-hexa-1,3,5-trienyl]-6-(4-nitro-phenyl)-cyclohexa-1,3-dienecarbaldehyde; 6-methyl-4-(4-methyl-pent-3-enyl)-6-thiophen-2-yl-cyclohexa-1,3-dienecarbaldehyde; 4-biphenyl-4-yl-6-methyl-6-(5-methyl-furan-2-yl)-cyclohexa-1,3-dienecarbaldehyde; 4,6-dihexyl-6-methyl-cyclohexa-1,3-dienecarbaldehyde; 4-hexyl-6-methyl-cyclohexa-1,3-dienecarbaldehyde; 4-biphenyl-4-yl-6-methyl-6-thiophen-2-yl-cyclohexa-1,3-dienecarbaldehyde; (E)-6,6-dimethyl-4-(2-(2,6,6-trimethylcyclohex-1-enyl)vinyl)cyclohexa-1,3-dienecarbaldehyde; 6-methyl-6-(5-methylfuran-2-yl)-4-(4-methylpent-3-enyl)cyclohexa-1,3-dienecarbaldehyde; 6,6-dimethyl-4-(5-methylfuran-2-yl)cyclohexa-1,3-dienecarbaldehyde; 6-methyl-4-(naphthalen-2-yl)cyclohexa-1,3-dienecarbaldehyde; (E)-4-(4,8-dimethylnona-3,7-dienyl)-6-methylcyclohexa-1,3-dienecarbaldehyde; 5,6-dimethyl-4-(thiophen-2-yl)cyclohexa-1,3-dienecarbaldehyde; 5,6,6-trimethyl-4-(thiophen-2-yl)cyclohexa-1,3-dienecarbaldehyde; 6-(biphenyl-4-yl)-4,6-dimethylcyclohexa-1,3-dienecarbaldehyde; 6,6-dimethyl-4-(naphthalen-2-yl)cyclohexa-1,3-dienecarbaldehyde; 4-(biphenyl-4-yl)-6,6-dimethylcyclohexa-1,3-dienecarbaldehyde; 4,6,6-trimethylcyclohexa-1,3-dienecarbaldehyde; 5,6-dimethyl-4,6-di(thiophen-2-yl)cyclohexa-1,3-dienecarbaldehyde; 6-methyl-4,6-diphenylcyclohexa-1,3-dienecarbaldehyde; 6-methyl-4,6-di(naphthalen-2-yl)cyclohexa-1,3-dienecarbaldehyde; 4,6-bis((E)-4,8-dimethylnona-3,7-dienyl)-6-methylcyclohexa-1,3-dienecarbaldehyde; cis-4-methyl-5-(3-methyl-but-2-enyl)-6-phenyl-cyclohexa-1,3-dienecarbaldehyde; trans-4-methyl-5-(3-methyl-but-2-enyl)-6-phenyl-cyclohexa-1,3-dienecarbaldehyde; cis and trans-6-(4-dimethylamino-phenyl)-4-methyl-5-(3-methyl-but-2-enyl)-cyclohexa-1,3-dienecarbaldehyde; 6-(4-(dimethylamino)phenyl)-4-methylcyclohexa-1,3-dienecarbaldehyde; 4-methyl-6-(4-nitrophenyl)cyclohexa-1,3-dienecarbaldehyde; 4-methylcyclohexa-1,3-dienecarbaldehyde; 4-hexyl-6-phenylcyclohexa-1,3-dienecarbaldehyde; 6-(4-(dimethylamino)phenyl)-4-hexylcyclohexa-1,3-dienecarbaldehyde; 4,6-diphenylcyclohexa-1,3-dienecarbaldehyde; 6-(4-(dimethylamino)phenyl)-4-phenylcyclohexa-1,3-dienecarbaldehyde; 6-(4-nitrophenyl)-4-phenylcyclohexa-1,3-dienecarbaldehyde; 4-(naphthalen-2-yl)-6-phenylcyclohexa-1,3-dienecarbaldehyde; 6-(4-(dimethylamino)phenyl)-4-(naphthalen-2-yl)cyclohexa-1,3-dienecarbaldehyde; 4-(naphthalen-2-yl)-6-(4-nitrophenyl)cyclohexa-1,3-dienecarbaldehyde; 4-(naphthalen-2-yl)cyclohexa-1,3-dienecarbaldehyde; (E)-4-(4,8-dimethylnona-3,7-dienyl)cyclohexa-1,3-dienecarbaldehyde; (E)-4-(4,8-dimethylnona-3,7-dienyl)-6-phenylcyclohexa-1,3-dienecarbaldehyde; (E)-6-(4-(dimethylamino)phenyl)-4-(4,8-dimethylnona-3,7-dienyl)cyclohexa-1,3-dienecarbaldehyde; 5-methyl-6-(4-nitrophenyl)-4-(thiophen-2-yl)cyclohexa-1,3-dienecarbaldehyde; 6-(4-(dimethylamino)phenyl)-5-methyl-4-(thiophen-2-yl)cyclohexa-1,3-dienecarbaldehyde; 5-methyl-6-phenyl-4-(thiophen-2-yl)cyclohexa-1,3-dienecarbaldehyde; 5-methyl-4-(thiophen-2-yl)cyclohexa-1,3-dienecarbaldehyde; 5-(4-methyl-pent-3-enyl)-4′-nitro-biphenyl-2-carbaldehyde; 4″-dimethylamino-[1,1′;3′,1″]terphenyl-4′-carbaldehyde; 4′″-dimethylamino-[1,1′;4′,1″;3″,1′″]quaterphenyl-4″-carbaldehyde; 4′-dimethylamino-5-(9H-fluoren-2-yl)-biphenyl-2-carbaldehyde; 4′-(dimethylamino)-5-methylbiphenyl-2-carbaldehyde; 5-(9H-fluoren-2-yl)-biphenyl-2-carbaldehyde; or 5-(4-methyl-pent-3-enyl)-biphenyl-2-carbaldehyde. In particular embodiments, the cyclohexadienal of formula (IV) may be 4-(biphenyl-4-yl)-6-methyl-6-(5-methylfuran-2-yl)cyclohexa-1,3-dienecarbaldehyde. In particular embodiments, the cyclohexadienal of formula (IV) may be 4,6-dihexyl-6-methylcyclohexa-1,3-dienecarbaldehyde. In particular embodiments, the cyclohexadienal of formula (IV) may be 6-(4-(dimethylamino)phenyl)-4-(naphthalen-2-yl)cyclohexa-1,3-dienecarbaldehyde. The cyclohexadienal of formula (IV) may be substantially pure, as defined herein. The present invention also contemplates a pharmaceutical composition comprising a cyclohexadienal, such as a cyclohexadienal of formula (IV).

Certain cyclohexadienals of the present invention may aromatize, a phenomenon known to those of skill in the art. In certain embodiments, these aromatized products are fluorescent. Accordingly, the present invention contemplates a method of preparing a fluorescent compound, comprising allowing a cyclohexadienal as described herein to aromatize. The aromatization may occur spontaneously, or in the presence of one or more external stimuli, such as heat, light, or reagents, as known to those of skill in the art. Such stimuli may include potassium permanganate and acidic conditions. Cyclohexadienals that are susceptible to aromatization may, in certain embodiments, comprise an aryl substituent, such as phenyl group. In certain methods that involve production of a fluorescent compound as described herein, the cyclohexadienal is further defined as 4-(4-methyl-pent-3-enyl)-6-(4-nitro-phenyl)-cyclohexa-1,3-dienecarbaldehyde. In certain methods that involve production of a fluorescent compound as described herein, the cyclohexadienal is further defined as 4-(4-methylpent-3-enyl)-6-phenylcyclohexa-1,3-dienecarbaldehyde.

Certain cyclohexadienals of the present invention may stimulate neurite outgrowth. Accordingly, the present invention contemplates a method of stimulating neurite growth, comprising administering to a cell an effective amount of a cyclohexadienal as described herein. The cyclohexadienal may be a compound of formula (IV), in certain embodiments. The cyclohexadienal of formula (IV) that stimulates neurite outgrowth may comprise a substituent selected from the group consisting of naphthyl, biphenyl and farnesyl, in certain embodiments, and such a substituent may be positioned at, e.g., R₅ or R₆. Cyclohexadienals employed in neurite outgrowth methods may, in certain embodiments, be selected from the group consisting of 4-(biphenyl-4-yl)-6-methyl-6-(5-methylfuran-2-yl)cyclohexa-1,3-dienecarbaldehyde, 4,6-dihexyl-6-methylcyclohexa-1,3-dienecarbaldehyde and 6-(4-(dimethylamino)phenyl)-4-(naphthalen-2-yl)cyclohexa-1,3-dienecarbaldehyde.

As used herein, “α,β-unsaturated aldehyde” refers to an aldehyde containing a double bond spanning carbon centers C2-C3.

As used herein, “β-substituted α,β-unsaturated aldehyde” refers to an aldehyde containing a double bond spanning carbon centers C2-C3 and a substituent at C3.

As used herein, the term “alkyl,” alone or in combination, refers to a straight- or branched-chain, single-, double- (alkenyl) or triple-bonded (alkynyl) alkyl radical containing from 1 to 30 carbon atoms. Examples of such radicals include, but are not limited to, methyl, ethyl, n-propyl, isopropyl, n-butyl, isobutyl, sec-butyl, tert-butyl, pentyl, iso-amyl, hexyl, decyl, and the like, wherein any one or more of the carbon-carbon bonds in such radicals may be double- or triple-bonded (e.g., but-2-enyl, 2-methylbut-2-enyl, hexa-2,4-dienyl, etc.). An alkyl radical may be substituted or unsubstituted. “Unsubstituted alkyl” refers to alkyl radicals consisting only of carbon and hydrogen. Alkyl radicals may be substituted (“substituted alkyl”) with groups other than hydrogen or hydrocarbons, such as aryl, amino, halogen, cyano, imino, nitro, thio (e.g., thioether, sulfhydryl), oxy (e.g., ether, hydroxy), alkoxy, carboxy, oxocarboxy, phosphino, or any combination thereof. A “lower alkyl” refers to an alkyl radical of 1-15 carbons. An “upper alkyl” refers to an alkyl radical of 16-30 carbons. In certain embodiments, compounds of the present invention entail only lower alkyl radicals or upper alkyl radicals.

As used herein, “cyclic alkyl” or “cycloalkyl” refers to a cyclic alkyl radical, wherein “alkyl” is defined above. Examples of such cycloalkyl radicals include, but are not limited to, cyclopropyl, cyclobutyl, cyclopentyl, cyclohexyl and the like. Cyclic alkyl radicals also encompass polycyclic alkyls (e.g., decahydronaphthalenyl, 1,2,3,4,4a,5-hexahydronaphthalene). A cycloalkyl radical may be unsubstituted or substituted. “Unsubstituted cycloalkyl” refers to cycloalkyl radicals consisting only of carbon and hydrogen. Cycloalkyl radicals may be substituted with groups other than hydrogen, such as alkyl, aryl, amino, halogen, cyano, imino, nitro, thio (e.g., thioether, sulfhydryl), oxy (e.g., ether, hydroxy), alkoxy, carboxy, oxocarboxy, phosphino, or any combination thereof. An “acyclic alkyl” is an alkyl group that is not cyclized.

As used herein, “aryl” to a carbocyclic aromatic radical or a heterocyclic aromatic radical, or a combination thereof (that is, two or more aryl groups may be fused). Non-limiting examples of aryl groups include phenyl, naphthyl, indenyl, indanyl, indolyl, azulenyl, fluorenyl, anthracenyl, furyl, thienyl, pyridyl, pyrrolyl, oxazolylyl, thiazolyl, imidazolyl and pyrazolyl. An aryl radical may be unsubstituted or substituted. “Unsubstituted aryl” refers to aryl radicals consisting only of carbon and hydrogen. Aryl radicals may be substituted with groups other than hydrogen, such as alkyl, aryl, amino, halogen, cyano, imino, nitro, thio (e.g., thioether, sulfhydryl), oxy (e.g., ether, hydroxy), alkoxy, carboxy, oxocarboxy, phosphino, or any combination thereof.

As used herein, a “cyclic moiety” refers to a cycloalkyl radical or an aryl radical, as defined above, or any combination thereof (that is, a cycloalkyl radical fused to an aryl radical, e.g., 1,2,3,4-tetrahydronaphthalenyl). A cyclic moiety may be unsubstituted or substituted. “Unsubstituted cyclic moiety” refers to a cyclic moiety that consists only of the ring atoms (which may consist of carbon and/or heteroatoms) and hydrogen.

As used herein, “halogen” refers to fluoro, chloro, bromo, or iodo.

As used herein, the term “amino,” alone or in combination, is used interchangeably with “amine” and refers to a primary (e.g., —NH₂), secondary (e.g., alkyl-NH—), tertiary (e.g., (alkyl)₂-N—), or quarternary (e.g., (alkyl)₃-N(+)—) amine radical.

In certain aspects, “derivative” refers to a chemically modified compound that still retains the desired effects of the compound prior to the chemical modification. Such derivatives may have the addition, removal, or substitution of one or more chemical moieties on the parent molecule. Non-limiting examples of the types modifications that can be made to the compounds and structures disclosed herein include the addition or removal of lower alkyls such as methyl, ethyl, propyl, or substituted lower alkyls such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, heteroatoms such as N, S, or O can be substituted into the structure instead of a carbon atom to generate, for example, a heterocycloalkyl structure. The present invention specifically contemplates employing L-proline derivatives in the methods described herein. In certain embodiments, a proline derivative is not D-proline.

As used herein, the term “effective” (e.g., “an effective amount”) means adequate to accomplish a desired, expected, or intended result.

In certain embodiments, the present invention provides for substantially pure cyclohexadienals. As used herein, the term “substantially pure” indicates that the cyclohexadienal of interest constitutes the predominant species in a mixture (that is, greater than 50%, or, in other words, at least 50% pure). A substantially pure cyclohexadienal may be about or at least about 51%, 55%, 60%, 65%, 70%, 75%, 80%, 85%, 90%, 95%, 99%, or 99.9% pure. In certain embodiments, a substantially pure compound excludes any naturally occurring mixtures that contain the compound. A substantially pure cyclohexadienal may also, or in the alternative, refer to the percent enantiomeric excess (% ee) of the compound. In certain embodiments, a substantially pure cyclohexadienal may exhibit a % ee of about or at least about 10%, 15%, 20%, 25%, 30%, 35%, 40%, 45%, 50%, 55%, 60%, 65% or higher, or any range derivable therein, such as about 10% to about 62%.

As used herein, “aromatize” refers to the tendency of certain compounds to transform from a non-aromatic compound to an aromatic compound over a period of time. The period of time may be from at least about, at most about, or about 1, 3, 5, 10, 20, 30, 40, 50, 60, or 90 minutes; 2, 3, 4, 5, 10, or 20 hours; 1, 2, 3, 4, 5, 6, or 7 days; or 2, 3, or 4 weeks or more, or any range derivable therein. Such transformation may take place naturally, or via exposure to additional stimulation, such as light, heat, and/or reactants. Such reactants may comprise, for example, potassium permanganate and acidic conditions (e.g., a pH of about or at most about 6.9, 6.5, 6.0, 5.5, 5.0, 4.5, 4.0, 3.5, 3.0, 2.5, 2.0, 1.5, 1.0 or 0.5).

Compounds of the present invention may contain one or more asymmetric centers and thus can occur as racemates and racemic mixtures, single enantiomers, diastereomeric mixtures and individual diastereomers. All possible stereoisomers of the compounds of the present invention are contemplated as being within the scope of the present invention. The chiral centers of compounds of the present invention can have the S— or the R-configuration, as defined by the IUPAC 1974 Recommendations. The present invention is meant to comprehend all such isomeric forms of the compounds of the invention.

The claimed invention is also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like. The term “pharmaceutically acceptable salts” refers to salts prepared from pharmaceutically acceptable non-toxic bases or acids including inorganic or organic bases and inorganic or organic acids, and are described in more detail herein.

Prodrugs and solvates of compounds of the present invention are also contemplated herein. The term “prodrug” as used herein, is understood as being a compound which, upon administration to a subject, such as a mammal, undergoes chemical conversion by metabolic or chemical processes to yield a compound any of the formulas herein, or a salt and/or solvate thereof (Bundgaard, 1985). Solvates of the compounds of the present invention include, but are not limited to, hydrates.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

FIG. 1. The self-condensation products of all-E-retinal (A) and citral (B).

FIG. 2. Non-limiting examples of homodimer cyclohexadienals produced according to the present invention.

FIG. 3. Non-limiting examples of heterodimer cyclohexadienals produced according to the present invention.

FIG. 4. Non-limiting examples of heterodimer cyclohexadienals produced according to the present invention.

FIG. 5. Non-limiting examples of heterodimer cyclohexadienals produced according to the present invention.

FIG. 6. Non-limiting examples of heterodimer cyclohexadienals produced according to the present invention.

FIG. 7. Non-limiting examples of heterodimer cyclohexadienals produced according to the present invention.

FIG. 8. Time course analysis of retinal homodimerization by NMR spectroscopy.

FIG. 9. Results of neurite outgrowth assay using certain compounds of the present invention (see Example 8).

FIG. 10. Anti-cancer assay results of certain compounds of the present invention (see Example 9).

FIGS. 11-14. Anti-bacterial assay results of certain compounds of the present invention (see Example 10).

FIGS. 15-16. Anti-fungal assay results of certain compounds of the present invention (see Example 10).

### DESCRIPTION OF ILLUSTRATIVE EMBODIMENTS

The present invention is based on the finding that L-proline and certain derivatives thereof catalyze the reaction of α,β-unsaturated aldehydes with β-substituted α,β-unsaturated aldehydes. Proline catalysts as described herein are relatively inexpensive and/or easily accessible. Methods of the present invention may employ a single aldehyde to provide a homodimer cyclohexadienal via self-condensation, or two specific aldehydes may be reacted to produce a heterodimer cyclohexadienal. The present invention is also drawn to the cyclohexadienals produced via the methods described herein. In certain embodiments, cyclohexadienals of the present invention are natural products or derivatives of natural products. In certain embodiments, cyclohexadienals of the present invention will find use as fluorescent probes, anti-cancer agents, anti-bacterial agents, and/or anti-fungal agents.

### A. PROLINE AS CATALYST

L-Proline is a widely employed organocatalyst as it is readily available and inexpensive. (DE 2102623; Eder et al., 1971; Dalko and Moisan, 2004; Seayad, 2005) (e.g., roughly $0.50/g, Sigma-Aldrich Co., Milwaukee, Wis.). It has been employed in a variety of asymmetric organic reactions including aldol (List, 2002; Alcaide and Almendros, 2003; Notz et al., 2004; List, 2004; Sakthivel et al., 2001; Córdova et al., 2002; Northrup and MacMillan, 2002; Bøgevig et al., 2002; Northrup et al., 2004; List et al., 2004; Casas et al., 2005), Diels Alder (Thayumanavan et al., 2002; Ramachary et al., 2002; Lakner and Negrete, 2002; Sundén et al., 2005; Ishihara and Nakano, 2005; Sabitha et al., 2005), Michael addition (Krause and Hoffmann-Röder, 2001; Christoffers and Baro, 2003; Horstmann et al., 2000; Halland et al., 2002; List et al., 2002; Halland et al., 2003; Betancort et al., 2004; Hechavarria Fonseca and List, 2004), among many others (Dalko and Moisan, 2004; Seayad and List, 2005).

Previous work has shown that the cyclohexadienal-producing reactions described herein can be promoted by trans-4-hydroxy-L-proline, D-proline and cis-4-hydroxy-D-proline (Asato et al., 1992). However, these reagents are relatively expensive and difficult to prepare (e.g., $5.00/g for trans-4-hydroxy-L-proline, $21/g for D-proline and $50.50/g for cis-4-hydroxy-D-proline, Sigma-Aldrich Co., Milwaukee, Wis.). Thus, the use of L-proline, which is readily accessible and relatively inexpensive (roughly $0.50/g) is an attractive alternative, particularly from a commercial standpoint.

Generally, the present invention contemplates the use of L-proline and its derivatives as a chiral auxiliary to catalyze asymmetric self-condensation of a variety of α,β-unsaturated aldehydes. The scope of the reaction was investigated with different substrates and proline catalysts and the reaction conditions were optimized. NMR and mass spec analyses as well as reactions with various proline derivatives also provided mechanistic insight into the reaction.

### B. SYNTHETIC METHODOLOGY

Initially, conditions were optimized for these reactions (varying the temperature and substrate to catalyst ratios), utilizing citral as a model substrate and L-proline to implement the reaction. Reactions mediated with other amino acids (Trp, His, Arg, Gly, and Ile) either fail or proceed to a lesser extent than proline (Asato et al., 1992). Product yields were highest when the reactions were carried out at room temperature versus −20-0° C. or 50-100° C. Citral dimer 1i is typically isolated as the sole product in 65% yield following 16-24 h incubation at room temperature (Table 1). This is in contrast to treatment of citral with NaH, which gives a mixture of products, 1i and 1i*, in a 5:95 product ratio (Scheme 1) (Taneja et al., 1988).

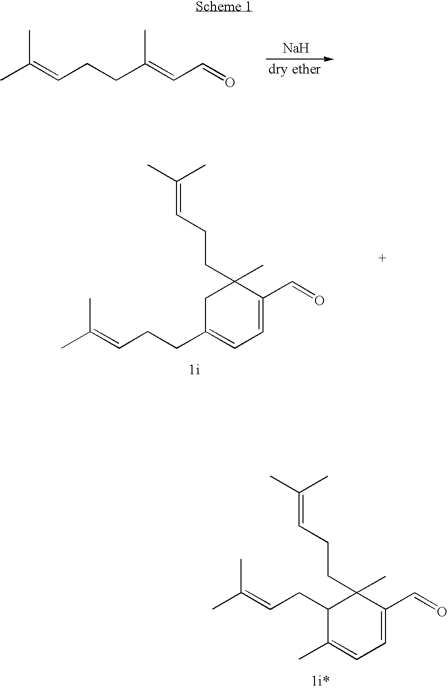

When the temperature is decreased (−20-0° C.), the reaction is sluggish and yields diminish to less than 5%. The majority of the starting material remains as its Schiff base at 48 h. On the contrary, when the temperature is increased, many unidentified byproducts begin to form within 1 h and citral dimer is obtained in less than 10% yield at 16 h. Varying the substrate to auxiliary agent (L-proline) ratios also had a dramatic effect on reaction yields. Six different substrate to catalyst ratios were studied: 1:0.5, 1:1, 1:1.25, 1:1.5, 1:1.75 and 1:2, respectively, whereby 1:1.5 molar concentrations exhibited the highest conversion rates (65%). At 1:0.5, 1:1 and 1:1.25 concentrations, the yields were <5%, 27% and 42%, respectively. Likewise, at higher proline concentrations, yields were reduced to 37% (1:1.75) and 35% (1:2).

TABLE 1
Effect of Various Substrates on Proline Promoted Homodimerization

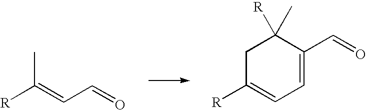

| | | | ^{c}ee |
|---|---|---|---|
| ^{a}R group | Compound | ^{b}yield | (%) |
| 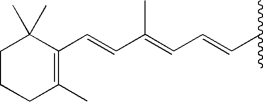 | 1a | 89 | 52 |
| 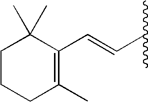 | 1b | 87 | 62 |
| 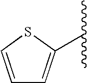 | 1c | 47 | 40 |
| 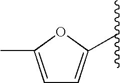 | 1d | 60 | 26 |
| 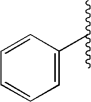 | 1e | 66 | 40 |
| 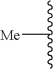 | 1f | 73 | 54 |
| 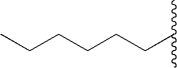 | 1g | 50 | 56 |
| 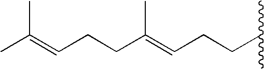 | 1h | 42 | 51 |
| 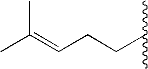 | 1i | 65 | 50 |
| 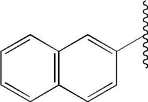 | 1j | 57 | 42 |
| 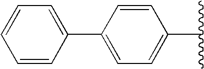 | 1k | 52 | 36 |
| 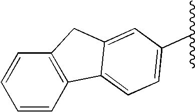 | 1l | 62 | 46 |

^{a}Reaction conditions: 1 equivalent of aldehyde and 1.5 equivalents of L-proline dissolved in ethanol and stirred at room temperature for 16–24 h.
^{b}Isolated yield,
^{c}Determined by Pr(hfc)₃ chiral shift reagent

Various substrates were tested utilizing these optimized conditions to investigate the generality of the reaction as illustrated in Table 1. Product yields ranged from 42-89% and ee's from 26-62%. Treatment of retinal (1a, requires added triethylamine) (Asato et al., 1992), and β-ionylideneacetaldehyde (1b) with ethanolic proline gave correspondingly higher yields than that originally reported and yields are provided for reaction with senecioaldehyde (1f) and citral (1i) that were not previously provided (Asato et al., 1992). The new substrates examined α,β-unsaturated aldehydes with conjugated chains (1a and 1b), the aromatic aldehydes, thiophene (1c) methylfuran (1d), and phenyl (1e), the aliphatic alkyl substituents (1g and 1h), as well as bulky substituents such as naphthalene (1j) biphenyl (1k) and fluorene (1l). Interestingly, thiophene-substituted (1c) gave an unexpectedly lower yield than methylfuran-substituted (1d) but gave correspondingly higher ee's. Modest yields were obtained with alkyl substituents (1g and 1h), presumably attributed to the flexibility of the groups versus the more rigid conjugated long chain substituents (1a) and (1b) where the highest yields were observed. While the naphthalene (1j), biphenyl (1k) and fluorene (1l) substituents were expected to result in low transformation efficiences due to steric hindrance (attributed to the bulkiness of the side chains), product yields (1j, 57%; 1k, 52%; 1l, 62%) were remarkably good, comparable to that of others examined. Circular dichroism (CD) analysis of the methylhydrazone derivatives of C-30 dimer (formed through dimerization of 1b) suggests that the absolute configuration of the major isomer formed in these L-proline mediated reactions is in the S-configuration as determined by the quadrant rule (chiral excition theory) (Harada and Nakanishi, 1972).

Since the reaction appeared quite universal, i.e. amenable to a variety of different substrates, various proline derivatives were analyzed to determine their effects on the enantioselectivity of the reaction. Previous work has shown that the reaction can be promoted by trans-4-hydroxy-L-proline, D-proline, and cis-4-hydroxy-D-proline (Asato et al., 1992). Citral (1i) was reacted with six different proline derivatives including L-proline methylester (2a), (Kyba et al., 1978) isopropyl ester (2b), (Kyba et al., 1978) benzyl ester (2c), (Kyba et al., 1978) prolinol (2d), diethyl prolinol (2e), (Zhou et al., 2004) and biphenyl prolinol (2f), (Zhou et al., 2004) Table 2. However, little enantiomeric selectivity (less than 20%) was observed in each case. The highest selectivity was obtained with α,α-diethyl prolinol 2e, which gave an enantiomeric excess of 15.5%. Reaction yields were moderate with the exception of 2c and 2d, which generated yields similar to that of L-proline. Solvent effects on citral dimerization were also examined with prolinol (2d); THF, DMSO, DMF, and isopropanol were each examined but no improvement on enantiomeric selectivity was observed. As with the proline assisted reaction, ethanol generated the highest yields (78%).

TABLE 2
Effect of Proline Derivatives on Homodimerization
hl,1

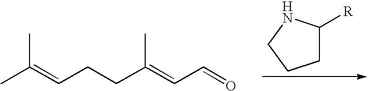

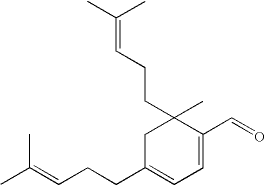

| ^{a}R group | catalyst | time (h) | ^{b}yield (%) |
|---|---|---|---|
| 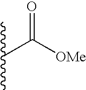 | 2a | 5 | 38 |
| 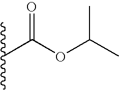 | 2b | 34 | 43 |
| 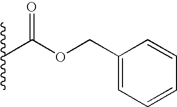 | 2c | 33 | 72 |
| 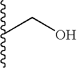 | 2d | 23 | 78 |
| 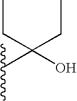 | 2e | 18 | 57 |
| 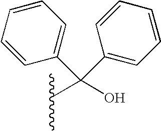 | 2f | 30 | 49 |

^{a}Reaction conditions: 1 equivalent of citral and 1.5 equivalents of catalyst dissolved in ethanol and stirred at room temperature for 16–24 h.
^{b}Isolated yield

Without wishing to be bound by theory, the inventors suggest that the proline promoted reaction ensues by nucleophilic attack of the aldehyde with proline, resulting in the formation of a Schiff base 3 (Scheme 2). Tautomerization (invoked by deprotonation of the β-methyl group) gives the β-methylenic proline adduct 4 that can be visualized to dimerize with 3 following a Diels-Alder- or Michael-like imine addition mechanism.

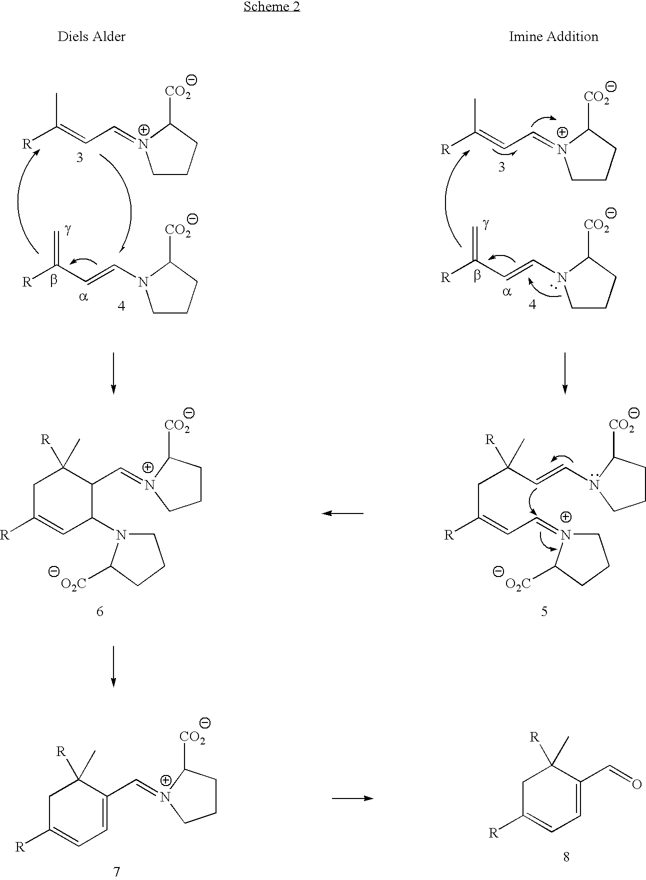

In certain methods of the present invention, certain equivalents of reagents are required. In certain embodiments, 1.5 equivalents of chiral auxiliary (e.g., L-proline) are needed in the reaction. Again, without wishing to be bound by theory, the inventors posit that this is presumably necessary to convert all of the starting aldehyde to that of the Schiff base (accounting for 1 equivalent of L-proline), while the additional 0.5 equivalents is needed to deprotonate the β-methyl group. In support of this mechanism, many of the proline derivatives listed in Table 2 were capable of catalyzing the reaction in similar yields to that of L-proline. Therefore, proton abstraction is likely not an intramolecular process involving that of the proline carboxylate.

Additionally, while proline derivatives have been reported to give modest improvements in enantioselectivity over proline itself, (Dalko and Moisan, 2004; Seayad and List, 2005) here, no improvement was observed. Hence, the reactive center might be too remote for these derivatized proline chiral auxiliary agents to impart much of an impact. This in turn would suggest that the reaction likely proceeds through imine addition versus a Diels-Alder based mechanism (Verdegem et al., 1997; Taneja et al., 1988; Valla et al., 2000; Yamad et al., 1973a; Yamad et al., 1973b; Duhamel et al., 1991); the Diels-Alder approach involves the enamine and γ-positions of the diene (Scheme 2). On the contrary, the Michael-like imine addition involves only the γ-position, a remote carbon center, of the cis-diene. The effect of the chiral auxiliary should be quite pronounced if the reaction followed a Diels-Alder reaction pathway. In further support of this notion, previous reports of proline-assisted Michael additions, where effects on ee's were observed, have involved addition by the α-position of the donor molecule (Scheme 3) (Krause and Hoffmann-Roder, 2001; Christoffers and Baro, 2003; Horstmann et al., 2000; Halland et al., 2002; List et al., 2002; Halland et al., 2003; Betancort et al., 2004; Hechavarria Fonseca and List, 2004). Here, we are two carbon atoms removed (addition occurs with the γ-carbon of the donor molecule) and the chiral auxiliary is too far removed to elicit an effect.

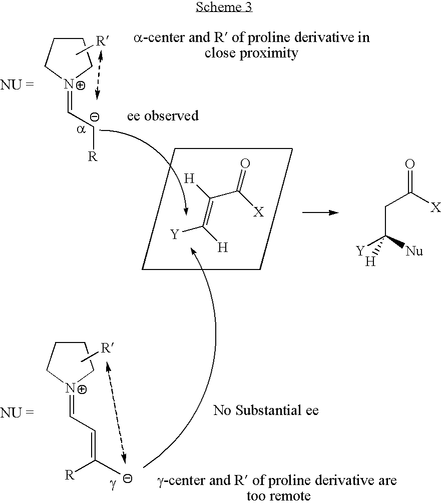

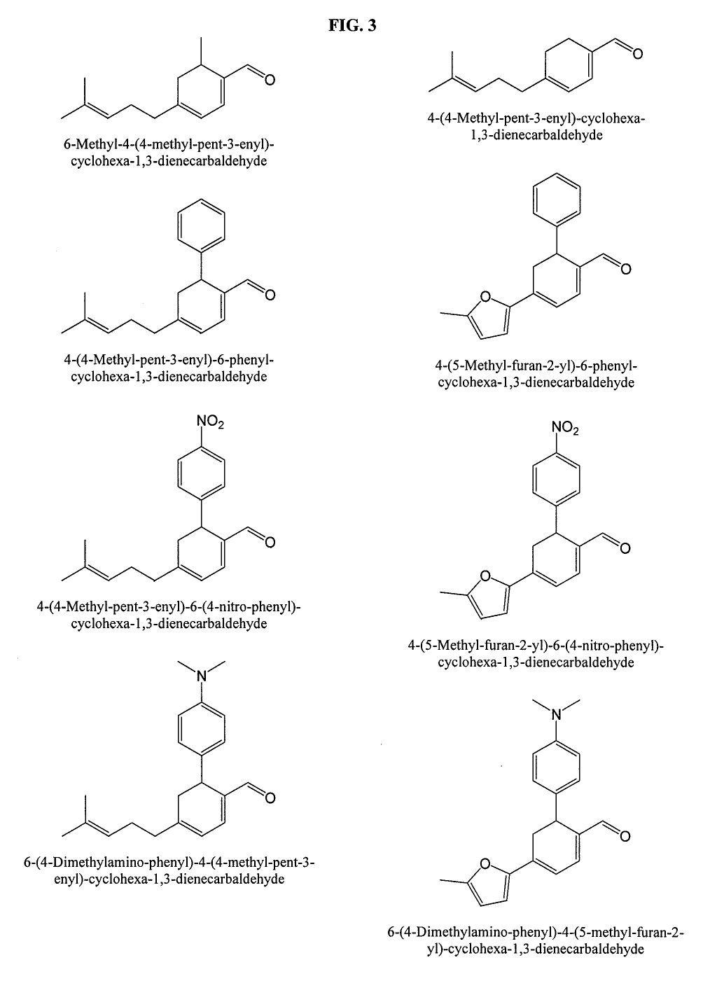

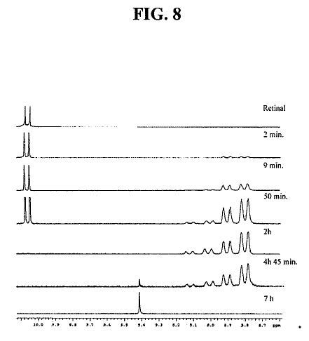

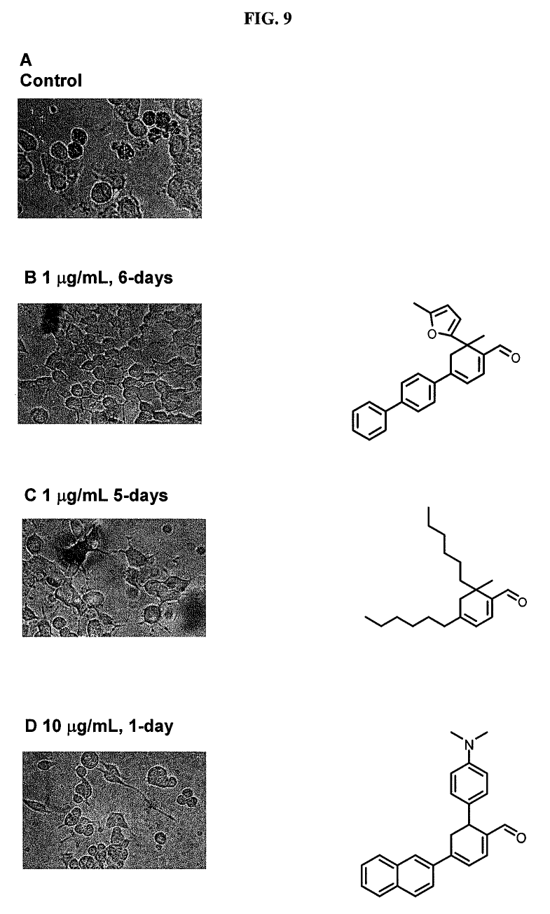

To provide additional experimental evidence for our proposed mechanism, we performed a time course analysis of this proline assisted dimerization reaction by NMR spectroscopy and mass spectrometry. The results are provided here for the homodimerization of retinal 1a (FIG. 8). Retinal and proline were mixed in an NMR tube. An immediate scan (2 min.) revealed formation of the Schiff base (3), giving both cis and trans isomers (α-carbon to the protonated imine) at 8.90 and 8.80 ppm respectively (Rabiller and Danho, 1984). After 9 min., two new pairs of peaks began to appear corresponding to cis/trans isomers (9.01 and 9.11 ppm, respectively) of intermediate 7. While full characterization of 7 could not be obtained due to convolution of the spectra, two additional pairs of doublets (7.25 ppm and 7.57 ppm) were detected corresponding to the coupled cyclohexadiene ring protons. Coupling of the protons were confirmed by H,H—COSY (provided as supplemental data). Retinal was fully consumed by 1 h 20 min. post-induction with intermediates 3 and 7 remaining, providing evidence that the condensation reaction occurs between two proline adducts (FIG. 3, condensation between 3 and 4) as opposed to one proline adduct and one aldehyde. At 3 h, triethylamine (1 eq.) was added to facilitate dimer formation (Asato et al., 1992). Traces of the C-40 dimer product were apparent by 4 h. The full NMR spectra of these time points are provided as supplemental information. Mass spec analysis of the reaction was performed at four time points (30 min, 1 h 30 min, 2 h 30 min and 4 h 30 min). The data were fully consistent with the NMR results (FIG. 9, reaction progression at 2 h 30 min), showing detection of both the Schiff base 3 and intermediate 7. NMR and MS experiments were also performed with citral (without added triethylamine). The data paralleled that observed with retinal and are included as supplemental information.

Additionally, β-methyl substituted benzylidene aldehyde was synthesized in deuterated form with the intent of measuring a kinetic isotope effect for the homodimerization reaction. However, a deuterium exchange experiment revealed significant solvent exchange by the reaction. Citral was reacted with proline in deuterated methanol and examined by both mass spectrometry and NMR spectroscopy. Mass spec analysis revealed the presence of citral dimer labeled with 1 through up to a possible 10 deuteriums. NMR analysis/integration of the peaks confirmed the incorporation of deuterium at the aldehyde position as well as C-3, C-3′, C-3″, C-5, C-7, C-1″. The significant exchange observed during the course of this reaction hinders the ability to measure a kinetic isotope effect. Thus, ¹³C-NMR may be utilized to aid in distinguishing between a Diels Alder versus a Michael-like imine addition for this reaction.

### C. FLUORESCENT PROBE APPLICATIONS

The present inventors have also discovered that surprisingly, reactions of certain compounds of the present invention result in fluorescent cyclohexadienal compounds. For example, when certain cyclohexadienals of the present invention are excited by an ultraviolet light source, they exhibit emission wavelengths that result in fluorescence. Cyclohexadienals of the present invention which possess conjugated double bonds are particularly contemplated as having fluorescent properties. In certain embodiments, fluorescent compounds of the present invention comprise one or more conjugated biphenyls (see, e.g., Scheme 4).

Certain substituted phenyl substituted cyclohexadienals of the present invention spontaneously aromatize to give fluorescent molecules (e.g., over a period of one to several days), such as 4-(4-methyl-pent-3-enyl)-6-(4-nitro-phenyl)-cyclohexa-1,3-dienecarbaldehyde (see FIG. 3). Aromatization may also be invoked by reaction of a cyclohexadienal of the present invention, such as a phenyl-substituted cyclohexadienal, with potassium permanganate under acidic conditions and heat to yield a fluorescent product (see Scheme 4). As such, the present invention contemplates fluorescent cyclohexadienal-containing compounds.

Certain fluorescent compounds of the present invention may have applications as, for example, fluorescent probes, such as for medical diagnostic and/or imaging purposes. Fluorescent compounds of the present invention may be used to examine the binding affinity of a particular cyclohexadienal to a protein of interest, as the fluorescence of a molecule may change depending on its environment. In certain embodiments, a fluorescent cyclohexadienal of the present invention may be appended to other moieties of interest, including targeting ligands, proteins and receptors, and used as a fluorescent probe.

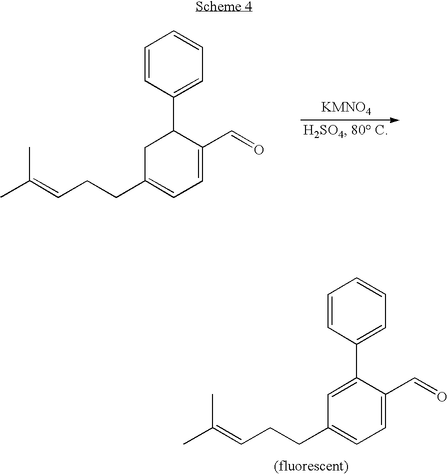

### D. OTHER BIOLOGICAL APPLICATIONS

In certain embodiments, cyclohexadienals of the present invention will find use as anti-cancer, anti-bacterial, anti-fungal, and/or anti-viral agents. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing tumor size, inhibiting tumor growth, reducing the blood supply to a tumor or one or more cancer cells, promoting an immune response against one or more cancer cells or a tumor, preventing or inhibiting the progression of a cancer, or increasing the lifespan of a subject with a cancer. An “anti-bacterial” agent is capable of negatively affecting bacterial growth, such as by killing bacteria or inhibiting their growth or replication. An “anti-fungal” agent is capable of negatively affecting fungal growth. An “anti-viral” agent is capable of inhibiting viral infection of cells.

1. Natural Products and Derivatives Thereof

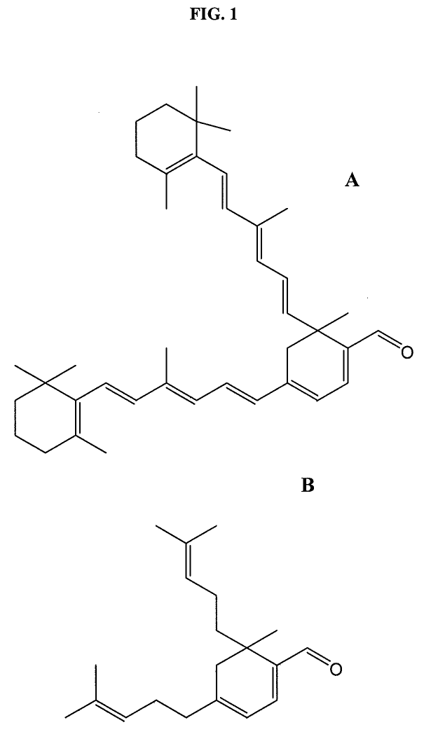

In certain embodiments, methods of the present invention permit access to natural products comprising a cyclohexadienal. For example, as discussed above, condensation of all-E-retinal gives a C-40 ring-fused dimer A shown in FIG. 1 (with a cyclohexadienal structural core) that has been implicated as a contributor of age-related macular degeneration, the leading cause of blindness in the elderly (Fishkin et al., 2005; Fishkin et al., 2004). While molecules of this structural scaffold are not unprecedented in nature, considerably less is known regarding the biological functions of these molecules. The self-dimerization of citral, for example, has been suspected since the late 1890's (Tiemann, 1898; Labbé, 1899). The 1,2,4-trisubstituted structure B (shown in FIG. 1) was definitively ascribed in 1932 (Fischer and Löwenberg, 1932). Recently, citral dimer B was isolated from the North Sea bryozoan Flustra foliacea and shown to exhibit anti-bacterial activity against Roseobacter sp. and Sulfitobacter sp. at 100 μg resulting in inhibition of 0.5 and 1.0 cm, respectively, via agar diffusion assay (Holst et al., 1994; Peters et al., 2003; Peters et al, 2004). In certain embodiments, the methods disclosed herein allow access to compounds such as A and B in transformations that involve easily-accessible, catalytic proline derivatives in the presence of mild reaction conditions. The present invention not only contemplates methods of synthesizing these cyclohexadienals, but the cyclohexadienals themselves as well.

2. Stimulators of Neurite Outgrowth

Certain compounds of the present invention have also been shown as stimulators of neurite outgrowth. The ability of neurons to extend neurites is of prime importance in establishing neuronal connections during development. It is also required during regeneration to re-establish connections destroyed as a result of a lesion. Several recognition molecules which act as molecular cues underlying promotion and/or inhibition of neurite growth have been identified (Martini, 1994). Molecules that stimulate neurite outgrowth activity could be important in the treatment of neurodegenerative disorders (e.g., Alzheimer's disease) and/or spinal cord injuries.

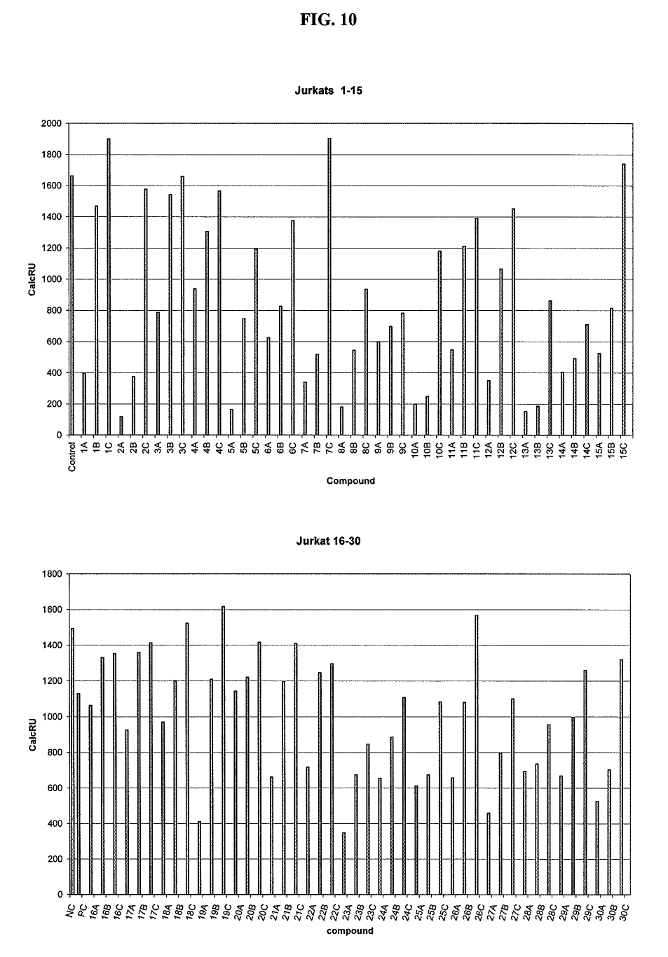

PC12 neuronal cells (from, e.g., American Type Culture Collection, 12301 Parklawn Drive, Rockville, Md., 20852 USA, ATCC Accession No. CRL 1721) are pluripotent and exhibit the ability to differentiate into neurons with the proper stimulation (e.g., neurite growth factor). The PC12 assay is a widely accepted method of identifying compounds that promote neurite outgrowth or survival (Green et al., 1998, specifically incorporated herein by reference). PC12 assay kits are available from various commercial sources (e.g., Millipore, Neurite Outgrowth Assay Kit, cat. no. NS225). Certain cyclohexadienals of the present invention have been found to stimulate neurite outgrowth in the PC12 assay. See, e.g., FIG. 10. Non-limiting examples of cyclohexadienals that stimulate neurite outgrowth include those comprising naphthyl, biphenyl and/or farnesyl substituents, or derivatives thereof (e.g., wherein the naphthyl, biphenyl and farnesyl substituents are further substituted with one or more groups, such as alkyl). Accordingly, in certain embodiments, the present invention contemplates cyclohexadienals that stimulate neurite outgrowth. In certain embodiments, cyclohexadienals of the present invention may find use in treatments of neurodegenerative disorders and/or spinal cord injuries.

### E. METHODS OF ADMINISTRATION

1. In Vitro Administration

The present invention contemplates administering a cyclohexadienal as disclosed herein to cells. Such administration may be via any method known to those of skill in the art. For example, administration may take place by incubation in or with (including immersion) a cyclohexadienal, perfusion or infusion with a cyclohexadienal, injection of the cells with a cyclohexadienal, or applying a cyclohexadienal to the cells or to a surface on which the tissue/organ lays and/or are in close proximity to.

2. In Vivo Administration

a. Pharmaceutical Formulations

The present invention also contemplates in vivo administration of cyclohexadienals as described herein. For example, a cyclohexadienal of the present invention may be comprised in a pharmaceutical composition. Pharmaceutical compositions of the present invention comprise an effective amount of one or more cyclohexadienals of the present invention, or additional agent, dissolved or dispersed in a pharmaceutically acceptable carrier. The phrases “pharmaceutical or pharmacologically acceptable” refer to molecular entities and compositions that do not produce an adverse, allergic or other untoward reaction when administered to an animal, such as, for example, a human, as appropriate. The preparation of an pharmaceutical composition that contains at least one cyclohexadienal of the present invention, or additional active ingredient, will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The cyclohexadienals of the present invention may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The cyclohexadienals of the present invention may be formulated into a composition in a free base, neutral, or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include cyclohexadienals of the present invention, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the cyclohexadienals of the present invention may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound (e.g., a cyclohexadienal of the present invention). In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

b. Combination Therapy

In order to increase the effectiveness of a cyclohexadienal of the present invention, the compounds of the present invention may be combined with traditional drugs. It is contemplated that this type of combination therapy may be used in vitro or in vivo. For example, an anti-cancer agent, an anti-bacterial agent, an anti-cancer agent, or an anti-viral agent may be combined with a cyclohexadienal. In a non-limiting example, an anti-cancer agent may be used in combination with a cyclohexadienal.

This process of combining agents may involve contacting the cell(s) with the agents at the same time or within a period of time wherein separate administration of the substances produces a desired therapeutic benefit. This may be achieved by contacting the cell, tissue or organism with a single composition or pharmacological formulation that includes two or more agents, or by contacting the cell with two or more distinct compositions or formulations, wherein one composition includes one agent and the other includes another.

The compounds of the present invention may precede, be co-current with and/or follow the other agents by intervals ranging from minutes to weeks. In embodiments where the agents are applied separately to a cell, tissue or organism, one would generally ensure that a significant period of time did not expire between the time of each delivery, such that the agents would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the candidate substance. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1, about 2, about 3, about 4, about 5, about 6, about 7 or about 8 weeks or more, and any range derivable therein, prior to and/or after administering the candidate substance.

Various combination regimens of the agents may be employed. Non-limiting examples of such combinations are shown below, wherein a cyclohexadienal is “A” and a second agent, such as an anti-cancer agent, is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

### F. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The following materials and methods were utilized in the Examples set forth below.

¹H and ¹³C NMR spectra were recorded on a Varian Inova-300 (300 MHz) spectrometer. Chemical shifts for ¹H and ¹³C NMR spectra are reported in ppm referenced to TMS (0 ppm) and coupling constants are reported in Hertz (Hz). Infrared (IR) spectra were generated on a Perkin-Elmer Spectrum One FT-IR spectrometer. Values are reported in wave number (cm⁻¹). HRMS determinations were performed with a PE SCIEX API QSTAR PULSAR mass spectrometer.

Fresh THF was dried by passage through an MBRAUN solvent purification system and stored over molecular sieves. All other solvents and reagents were purchased and used without further purification. Self-condensation products 1a (Thomas and Guntz-Dubin, 1976), 1b (Verdegem et al., 1997), and 1c (Asato et al., 1992) have been reported previously. Nitrilges 2c (Ramamurthy et al., 1975), 2d (McFarland et al., 1969), 2f (Huang et al., 1983), and 2g (DE 2456958) and α,β unsaturated aldehydes 3c (Ramamurthy et al., 1975), 3d (Meyers et al., 1983), 3e (Eberback and Roser, 1987), 3f (Ono et al., 1982; Syper, 1987), and 3g (DE 2439294) have been reported but not fully characterized. Additional spectroscopic information on these compounds is provided herein. Proline catalysts, 4a-c (Kyba et al., 1978), 4b (Kyba et al., 1978), and 4e-f (Zhou et al., 2004), have also been previously reported while 4d was purchased. Spectral information on nitrites 2e and 2h, aldehyde 3h, and ring-fused homodimers 1d to 1h are reported herein. Chromatographic separations were achieved by flash silica chromatography by EMD Silica Gel 60 (230-400 mesh).

### Example 1

### Synthesis of Certain Nitriles of the Present Invention

Nitriles 3b-3e, 3g, and 3j-3l were synthesized following literature protocols (Uchikawa et al., 2002) and purified by flash column chromatography (gradient of 2-10% ethyl acetate/hexanes).

3-Methyl-5-(2,6,6-trimethyl-cyclohex-1-enyl)-penta-2,4-dienenitrile (3b) (Ramamurthy et al., 1975): Yield: 94%: ¹H NMR (300 MHz, CDCl₃, 25° C.), δ: 6.54 (d, 1H, J=16.2 Hz), 6.11 (d, 1H J=16.2 Hz), 5.06 (s, 1H), 2.16 (d, 3H, J=0.6 Hz), 1.980-2.03 (m, 2H), 1.67 (d, 3H, J=0.9 Hz), 1.54-1.62 (m, 2H), 1.41-1.46 (m, 2H), 1.01 (s, 6H). ¹³C NMR (75 MHz, CDCl₃, 25° C.) δ: 157.1 (C), 136.1 (C), 135.6 (CH), 132.9 (CH), 130.2 (C) 117.9 (C), 96.6 (CH), 39.6 (CH₂), 34.3, (C), 33.3, (CH₂), 29.0 (2×CH₃), 21.8 (CH₂), 19.2 (CH₃), 16.5 (CH₃); IR (neat) v 2925, 2855,2210, 1738, 1614,1585, 1455, 1375, 1365, 1217, 966 cm⁻¹. HRMS (ESI) for C₁₅H₂₁NLi (M+Li)⁺: calcd 222.1834, found 222.1828.

### Example 2

### Synthesis of Certain α,β Unsaturated Aldehydes of the Present Invention

α,β-Unsaturated aldehydes were prepared by reduction of their corresponding nitrites (3b-3e, 3g and 3j-3l) and purified by flash column chromatography (2-10% ethyl acetate/hexanes) (Taber et al., 1987). Farnesal was synthesized from farnesol (Hu et al., 2004). In all cases, the all-trans isomer was utilized in self-condensation reactions.

3-Methyl-5-(2,6,6-trimethyl-cyclohex-1-enyl)-penta-2,4-dienal (4b) (Ramamurthy et al. 1975): Yield: 97%: ¹H NMR (300 MHz, CDCl₃, 25° C.), δ: 10.09 (d, 1H, J=8.1 Hz), 6.70 (d, 1H, J=16.2 Hz), 6.17 (d, 1H, J=15.9 Hz), 5.89 (d, 1H, J=7.8 Hz), 2.27 (s, 3H), 2.00 (t, 2H, J=6.0 Hz), 1.68 (s, 3H), 1.52-1.62 (m, 2H), 1.42-1.45 (m, 2H), 1.00 (s, 6H). ¹³C NMR (75 MHz, CDCl₃, 25° C.) δ: 191.1 (CH), 157.2, (C), 136.1 (C), 135.6 (CH), 132.8 (CH), 132.5 (C), 130.2 (CH), 39.6 (CH₂), 34.2 (C), 33.2 (CH₂) 28.9 (2×CH₃), 21.7 (CH₂), 19.1 (CH₃), 16.5 (CH₃); IR (neat) v 2930, 2865, 1738, 1665, 1606, 1448, 1376, 1364, 1216, 1206, 1116, 963, 764, 749,732 cm⁻¹;HRMS (ESI) for C₁₅H₂₂OLi (M+Li)⁺: calcd 225.1831, found 225.1835.

### Example 3

### Synthesis of Homodimer Self-Condensation Products

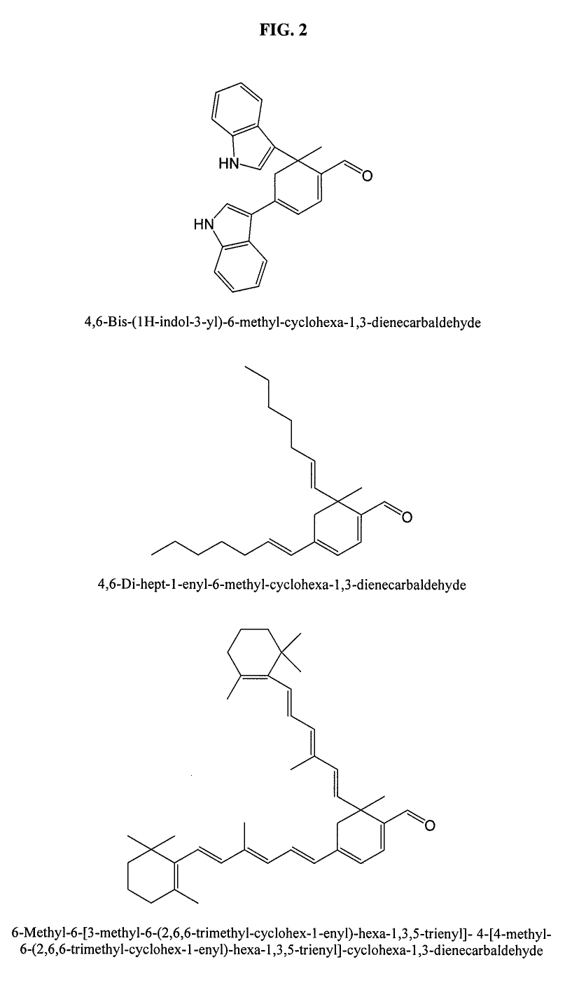

Ring-fused homodimers were generated by self-condensation of α,β-unsaturated aldehydes, modification of Asato et al. (1992). Certain homodimers prepared by methods of the present invention are shown in FIG. 2. The generalized approach is illustrated below for 6-methyl-4,6-di-thiophen-2-yl-cyclohexa-1,3-dienecarbaldehyde (1c).

6-Methyl-4,6-di-thiophen-2-yl-cyclohexa-1,3-dienecarbaldehyde (1c): To an oven-dried flask was added 3-thiophen-2-yl-but-2-enal (3c) (20 mg, 0.132 mmol) dissolved in 10 mL of 200 proof ethanol. To this solution, was added L-proline (23 mg, 0.200 mmol). The mixture was allowed to stir at RT for 24 h prior to quenching the reaction with D.I. H₂0 (30 mL) and extraction with hexanes (3×50 mL). The combined organics were washed with brine, dried over MgSO₄, and concentrated in vacuo. Purification by flash chromatography (5% ethyl acetate/hexanes) afforded 17.7 mg of 1c as a red oil (47%).

¹H NMR (300 MHz, CDCl₃, 25° C.), δ: 9.49 (s, 1H), 7.35-7.40 (m, 1H) 7.28-7.30 (m, 2H), 7.05-7.14 (m, 3H), 6.93 (d, 1H, J=6.0 Hz), 6.62 (dd, 1H, J=0.9, 6.3 Hz), 3.21 (d, 1H, J=17.4 Hz), 2.93 (dd, 1H, J=1.5, 17.4 Hz), 1.84 (s, 3H). ¹³C NMR (75 MHz, CDCl₃, 25° C.) δ: 192.0 (CH), 152.4 (C), 143.7 (CH), 142.2 (C), 141.5 (C), 138.8 (C), 128.6 (CH), 128.6 (CH), 128.5(CH), 127.9 (CH), 126.6 (CH), 126.2 (CH), 123.6 (CH), 45.2 (CH₂), 29.9 (C), 27.2 (CH₃). IR (neat) v 3006, 2928, 2855, 1738, 1455, 1365, 1217, 764 cm⁻¹. HRMS (ESI) for C₁₆H₁₄OS₂Li (M+Li)⁺: calcd 293.0646, found 293.0654.

### Example 4

### Synthesis of Certain Heterodimer Self-Condensation Products of the Present Invention

The present invention also encompasses the production of heterodimer cyclohexadienals via the proline mediated condensation of one β-methylenic and one α,β-unsaturated aldehydes.

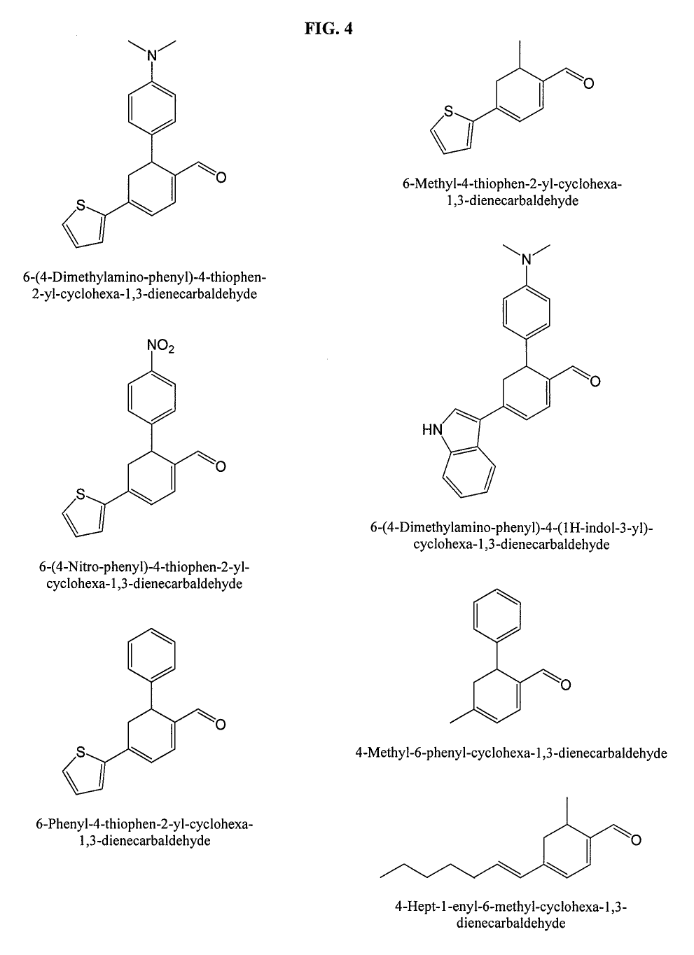

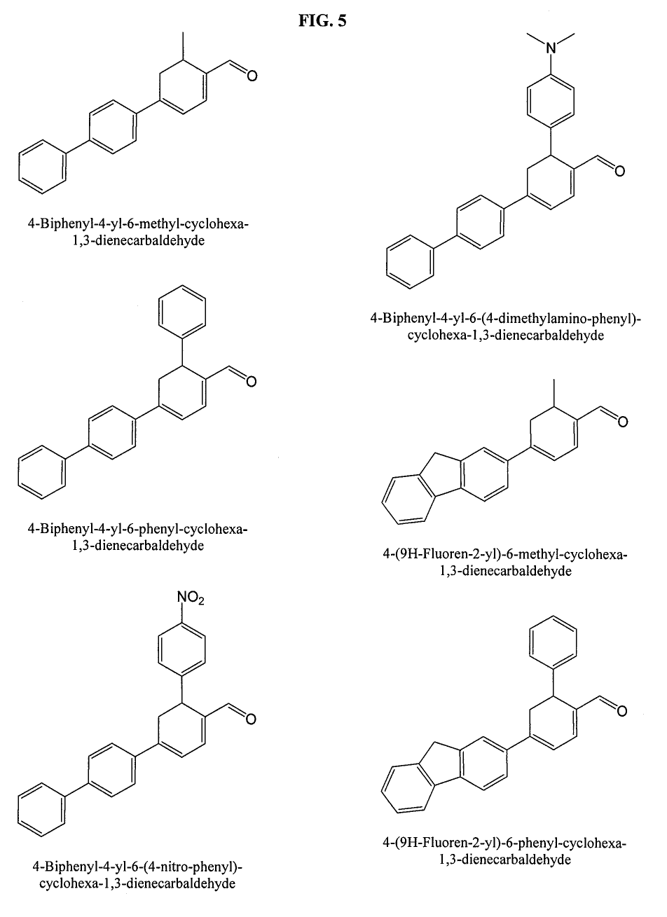

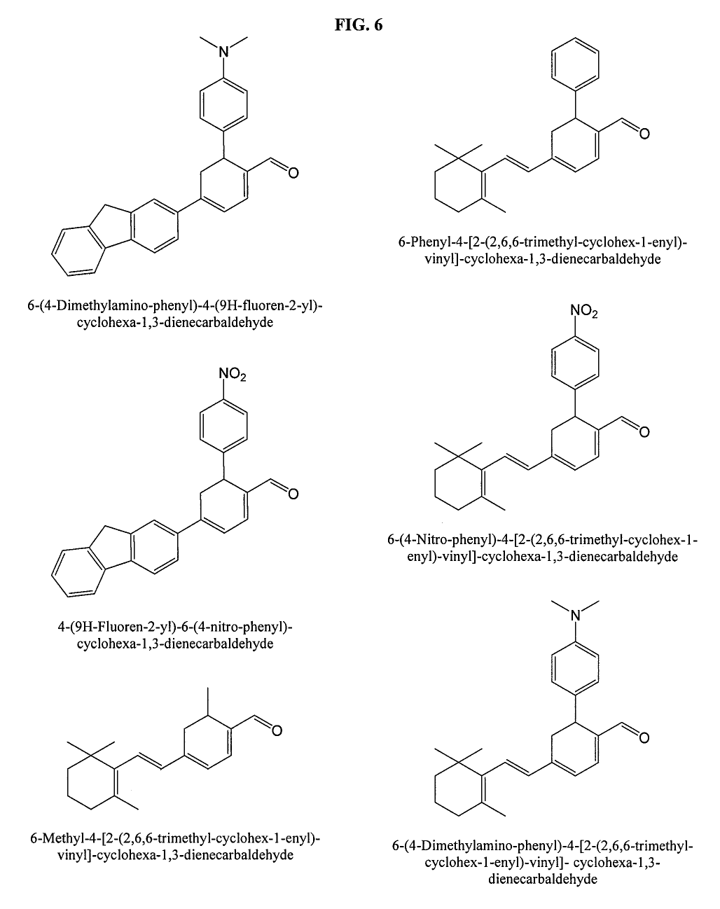

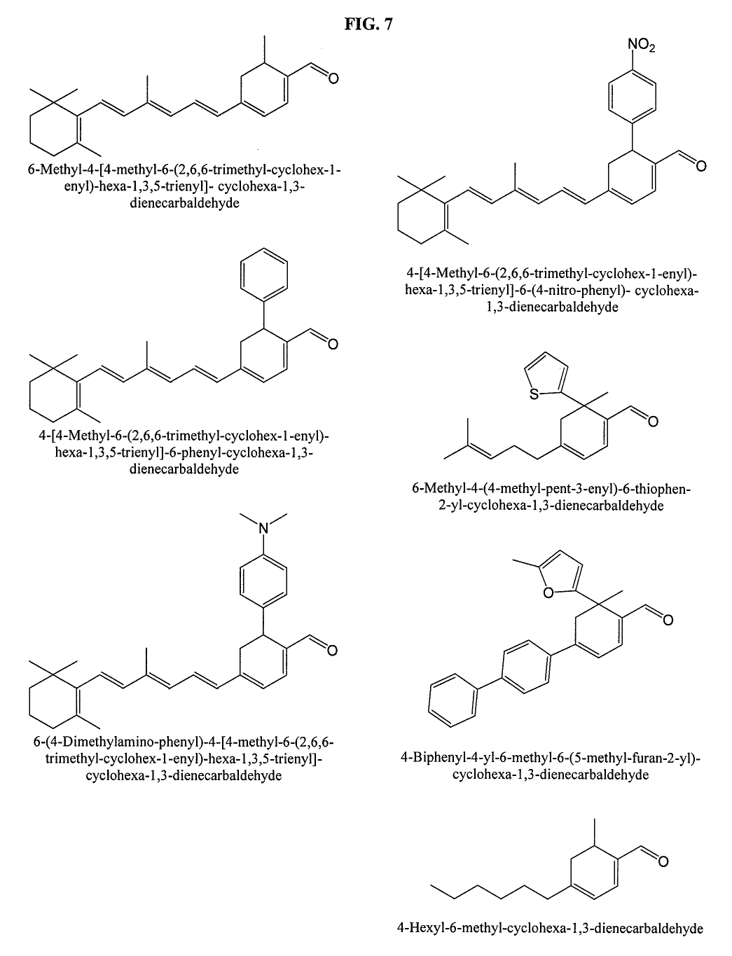

In an embodiment, (see Scheme 5 below) starting α,β-unsaturated aldehyde cinnamaldehyde (0.0253 g, 1.15 equivalents) was dissolved in ethanol (5 mL) with 3.4 equivalents (0.062 g) of proline. After about three hours, at which point Schiff base formation is complete, monitored via thin layer chromatography (TLC), one equivalent of β-methylenic aldehyde (E)-3-(5-methylfuran-2-yl)but-2-enal (0.025 g, 1 equivalent) was added to the reaction mixture and stirred at ˜25° C. for 18 hours. The reaction was monitored by (TLC). Upon completion, the reaction was quenched via the addition of water (20 mL) and the reaction mixture was extracted with a 1:1 ethyl acetate/hexanes solution and was washed with brine. The organic layer was dried over magnesium sulfate (MgSO₄) and concentrated under vacuum. The crude product was purified by column chromatography as described above for the homodimer cyclohexadienals to yield 4-(5-methylfuran-2-yl)-6-phenylcyclohexa-1,3-dienecarbaldehyde (0.0126 g, 28.6%). A number of such fused-ring heterodimers were similarly prepared, with the reaction time varying from about 16 to about 24 hours. Examples of heterodimer cyclohexadienals produced according to the present invention are shown in FIGS. 3-7.

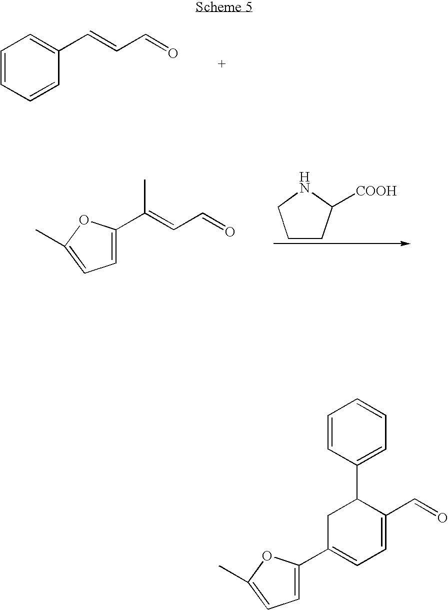

### Example 5

### Non-limiting Example of a Synthetic Method of the Present Invention

The present invention encompasses a method for preparing homodimer and heterodimer cyclohexadienals according to the method generally described by Scheme 6 below:

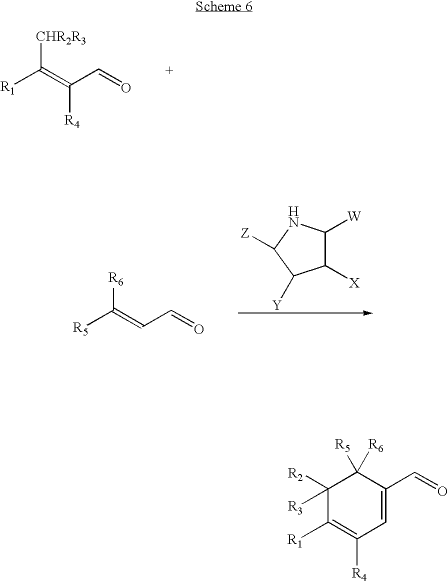

As described by Scheme 6, the various substituents may be varied as known to those skilled in the art. Specifically, the substituents W, X, Y and Z independently comprise, but are not limited to, H; halogen; —COOR_{A}, wherein R_{A} is selected from the group consisting of H, alkyl, cycloalkyl and aryl; —CR_{B}R_{C}R_{D}, wherein R_{B}, R_{C}, and R_{D} are each independently selected from the group consisting of H, alkyl, cycloalkyl, aryl, —CN and halogen, or wherein R_{B}, R_{C}, and/or R_{D} taken together are comprised in a cyclic moiety with C, or wherein R_{C} is not present; —C═N—R_{E}, wherein R_{E} is selected from the group consisting of alkyl, cycloalkyl and aryl; —NR_{F}R_{G}, wherein R_{F} and R_{G} are independently selected from the group consisting of H, alkyl, cycloalkyl and aryl, or wherein R_{F} and R_{G} taken together are comprised in a cyclic moiety with N; —N═R_{H}, wherein R_{H} is selected from the group consisting of alkyl, cycloalkyl and aryl; and —OR_{I}, wherein R_{I} is selected from the group consisting of alkyl, cycloalkyl and aryl. Aldehyde substituents R₁, R₂, R₃, R₄, R₅ and R₆ may each independently comprise, but are not limited to, H; halogen; —CR_{A}R_{B}R_{C}, wherein R_{A}, R_{B}, and R_{C} are each independently selected from the group consisting of H, alkyl, cycloalkyl, aryl, —CN and halogen, or wherein R_{A}, R_{B}, and/or R_{C} taken together are comprised in a cyclic moiety with C, or wherein R_{C} is not present; —C═N—R_{D}, wherein R_{D} is selected from the group consisting of alkyl, cycloalkyl and aryl; —NR_{E}R_{F}, wherein R_{E} and R_{F} are independently selected from the group consisting of H, alkyl, cycloalkyl and aryl, or wherein R_{E} and R_{F} taken together are comprised in a cyclic moiety with N; —N═R_{G}, wherein R_{G} is selected from the group consisting of alkyl, cycloalkyl and aryl; and —OR_{H}, wherein R_{H} is selected from the group consisting of alkyl, cycloalkyl and aryl. In certain embodiments, R₁ and R₂, R₁ and R₄, R₂ and R₃, and/or R₅ and R₆ may comprise a cyclic moiety. FIGS. 1-7 depict certain cyclohexadienals of the present invention.

### Conclusions in View of Examples 1-5

The results discussed in Examples 1-5 above demonstrate the use of proline to promote asymmetric self-condensation of α,β-unsaturated aldehydes to form trisubstituted cyclohexadiene products. Reaction conditions are mild and yet amenable to a variety of different substrates yielding molecules with complex scaffolds from simple precursors. The progress of the reaction was monitored, in time course analyses, by NMR spectroscopy and mass spectrometry providing evidence for the intermediacy of Schiff base 3 and protonated imine 7. Moreover, these experiments revealed the complete loss of starting aldehyde during the reaction in support of a two proline adduct based mechanism. Additionally, investigations with various proline derivatives gave similar yields to that of L-proline suggesting that deprotonation and activation of the β-methyl group to give 4 is an intermolecular process and does not involve the proline carboxylate of the Schiff base (3). The moderate ee's exhibited by these proline chiral auxiliaries is suggestive that the reaction likely proceeds through imine addition versus a Diels Alder based mechanism since a Diels-Alder would involve two reaction centers, the enamine and γ-positions of the diene, while imine addition would only involve the γ-position, a remote carbon center, of the cis-diene.

### Example 6

### NMR Analysis of Retinal Self-Condensation Reaction

A 5 mm NMR tube was filled with 800 μL of CD₃OD, placed in a 300 MHz NMR spectrometer. Once properly shimmed, another tube was filled with 500 μL of all-trans retinal (30 mg/mL, 0.05278 mmol) and the instrument reshimmed. The reaction was started by ejecting the all-trans retinal and adding 304 μL, 1.5 eq., of the L-proline solution (30 mg/mL, 0.07921 mmol), shaking the tube, reinserting the tube into the NMR, and immediately acquiring a spectrum that took a total time of 2 minutes. After the initial spectrum the instrument was periodically reshimmed. After 3 h 30 min., all of the all-trans retinal had been consumed and all that existed was Schiff base 3 and intermediate 7 at which time the NMR tube was removed and 180 μL of triethylamine (30 mg/mL, 0.5340 mmol) was added followed by shaking and reinsertion of the tube. After a few minutes, formation of the C-40 retinal dimer was present. After 2 hours 30 min., the reaction was complete. Spectra were acquired over an eight-hour period at various time points.

Mass spec analysis of the reaction was taken at 30, 90, 150, and 270 min respectively. Intermediates 3 (M+Li=388.2812 amu) and 7 (M+654.5181 amu) were both observed.

### Example 7

### Synthesis of a Fluorescent Compound of the Present Invention 5-(4-Methylpent-3-enyl)biphenyl-2-carbaldehyde

Cyclohexadiene is dissolved in 8 mL of 200 proof ethanol and is placed in a preheated hot oil bath adjusted to 80° C. This solution is stirred for five minutes before the addition of 2 equivalents of 1M potassium permanganate with enough sulfuric acid to bring the solution of a pH between 4 and 5. The solution is monitored via thin layer chromatography (TLC) until reaction is complete. The solution is then extracted with ethyl ether (3×50 mL). The organic layer is then washed with brine, dried over magnesium sulfate and concentrated in vacuo. The final product, 5-(4-methylpent-3-enyl)biphenyl-2-carbaldehyde, is then purified via flash chromatography with a mobile phase consisting of 10% ethyl acetate/hexane.

### Example 8

### Stimulation of Neurite Outgrowth by Certain Compounds of the Present Invention

PC12 cells were maintained in 100 mm Collagen Type IV dishes at 37° C. in a 5% CO₂/air temperature. The cell culture medium consisted of RPMI 1640, with 10% heat-inactivated horse serum and 5% fetal bovine serum. Cells were removed from the culture flasks by flushing with fresh culture medium. Cells were then transferred to a 15 mL falcon tube and centrifuged for 15 minutes at 1000 rpm. The medium was removed and the cells were resuspended in fresh culture medium. The cells were counted with a hemocytometer and replated in a 96 well Collagen Type IV plate at 10,000 cells/well with enough fresh medium to bring the volume to 200 μL. The cells are allowed to adhere for 24 hours before introduction of a test cycloehexadienal compound at three concentrations in duplicate, 100, 10 and 1 μg/mL respectively. Each well is visually monitored over a period of 2 weeks for neurite outgrowth and intercellular connections. Fresh medium is added when necessary. See FIG. 10.

### Example 9

### Anti-cancer Cytotoxicity of Certain Compounds of the Present Invention

Anti-cancer cytotoxicity assays were performed with Jurkat's, a human T-cell leukemia cell line. The cells were maintained in 75 cm² culture flasks at 37° C. under a 5% CO₂ atmosphere. The cell culture medium consisted of RPMI 1640 with 10% fetal bovine serum. Cells were harvested by centrifugation (20 minutes at 1000 rpm), resuspended in fresh culture media and counted on a hemocytometer. The cells (1 mL) were arrayed (200,000 cells/well) in 48 well plates to which compound was added at final concentrations of 100, 10 and 1 μg/mL respectively. Cells were incubated for 24 hours, transferred to 1.5 mL eppendorf tubes and centrifuged for 15 min. at 14000 rpm. The medium was removed and the cell pellet resuspended in 1 mL of phosphate buffer saline (PBS) to wash the cells. The cells were again centrifuged for 15 minutes at 14000 rpm. The PBS was removed and the cell pellet resuspended in a lysis buffer. The lysed cells were then placed in a black 96 well plate and analyzed using a Bio-Tek fluorimeter to measure relative fluorescence of propidium iodide binding to DNA. See FIG. 10, wherein the following compounds and concentrations were tested:
A=100 μg/mL
B=10 μg/mL
C=1 μg/mL
NC=negative control (no solvent)
PC=positive control (solvent used to dissolve compound alone)
1 6-methyl-4,6-di(naphthalen-2-yl)cyclohexa-1,3-dienecarbaldehyde
2 4-(naphthalen-2-yl)-6-phenylcyclohexa-1,3-dienecarbaldehyde
3 6-(4-(dimethylamino)phenyl)-4-(naphthalen-2-yl)cyclohexa-1,3-dienecarbaldehyde
4 6-methyl-4-(naphthalen-2-yl)cyclohexa-1,3-dienecarbaldehyde
5 3-Naphthalen-2-yl-but-2-enal (napthalene starting material)
6 6-methyl-4,6-diphenylcyclohexa-1,3-dienecarbaldehyde
7 6-methyl-4,6-diphenylcyclohexa-1,3-dienecarbaldehyde (deuterated species)
8 4-(5-Methyl-furan-2-yl)-6-phenyl-cyclohexa-1,3-dienecarbaldehyde
9 6-(4-Dimethylamino-phenyl)-4-(5-methyl-furan-2-yl)-cyclohexa-1,3-dienecarbaldehyde
10 6-(4-Dimethylamino-phenyl)-4-(4-methyl-pent-3-enyl)-cyclohexa-1,3-dienecarbaldehyde
11 4-Biphenyl-4-yl-6-(4-dimethylamino-phenyl)-cyclohexa-1,3-dienecarbaldehyde
12 6-Phenyl-4-[2-(2,6,6-trimethyl-cyclohex-1-enyl)-vinyl]-cyclohexa-1,3-dienecarbaldehyde
13 4,6-bis((E)-4,8-dimethylnona-3,7-dienyl)-6-methylcyclohexa-1,3-dienecarbaldehyde
14 6-Methyl-4,6-di-thiophen-2-yl-cyclohexa-1,3-dienecarbaldehyde
15 cis and trans-6-(4-Dimethylamino-phenyl)-4-methyl-5-(3-methyl-but-2-enyl)-cyclohexa-1,3-dienecarbaldehyde
16 4-(naphthalen-2-yl)-6-(4-nitrophenyl)cyclohexa-1,3-dienecarbaldehyde
17 4-(5-Methyl-furan-2-yl)-6-(4-nitro-phenyl)-cyclohexa-1,3-dienecarbaldehyde
18 4-(9H-Fluoren-2-yl)-6-phenyl-cyclohexa-1,3-dienecarbaldehyde
19 6-Methyl-4,6-bis-(4-methyl-pent-3-enyl)-cyclohexa-1,3-dienecarbaldehyde
20 6-Phenyl-4-thiophen-2-yl-cyclohexa-1,3-dienecarbaldehyde
21 6-(4-Dimethylamino-phenyl)-4-thiophen-2-yl-cyclohexa-1,3-dienecarbaldehyde
22 4-(9H-Fluoren-2-yl)-6-methyl-cyclohexa-1,3-dienecarbaldehyde
23 5-(4-Methyl-pent-3-enyl)-biphenyl-2-carbaldehyde
24 (E)-4-(4,8-dimethylnona-3,7-dienyl)-6-phenylcyclohexa-1,3-dienecarbaldehyde
25 6-(4-Dimethylamino-phenyl)-4-(4-methyl-pent-3-enyl)-cyclohexa-1,3-dienecarbaldehyde
26 6-(4-(dimethylamino)phenyl)-5-methyl-4-(thiophen-2-yl)cyclohexa-1,3-dienecarbaldehyde
27 6-(4-Nitro-phenyl)-4-thiophen-2-yl-cyclohexa-1,3-dienecarbaldehyde
28 6-(4-Nitro-phenyl)-4-[2-(2,6,6-trimethyl-cyclohex-1-enyl)-vinyl]-cyclohexa-1,3-dienecarbaldehyde
29 6-Methyl-4,6-bis-(5-methyl-furan-2-yl)-cyclohexa-1,3-dienecarbaldehyde
30 4-Biphenyl-4-yl-6-(4-nitro-phenyl)-cyclohexa-1,3-dienecarbaldehyde
31 4-methyl-6-(4-nitrophenyl)cyclohexa-1,3-dienecarbaldehyde
32 4′-(dimethylamino)-5-methylbiphenyl-2-carbaldehyde
33 4-Methyl-6-phenyl-cyclohexa-1,3-dienecarbaldehyde
34 4,6,6-trimethylcyclohexa-1,3-dienecarbaldehyde
35 6-(4-(dimethylamino)phenyl)-4-phenylcyclohexa-1,3-dienecarbaldehyde
36 4-Biphenyl-4-yl-6-(4-dimethylamino-phenyl)-cyclohexa-1,3-dienecarbaldehyde
37 4′-(dimethylamino)-5-methylbiphenyl-2-carbaldehyde
38 cis-4-Methyl-5-(3-methyl-but-2-enyl)-6-phenyl-cyclohexa-1,3-dienecarbaldehyde
39 4″-Dimethylamino-[1,1′;3′,1″]terphenyl-4′-carbaldehyde
40 (E)-6-(4-(dimethylamino)phenyl)-4-(4,8-dimethylnona-3,7-dienyl)cyclohexa-1,3-dienecarbaldehyde
41 4,6-diphenylcyclohexa-1,3-dienecarbaldehyde
42 6-(4-nitrophenyl)-4-phenylcyclohexa-1,3-dienecarbaldehyde
43 6-Methyl-4-thiophen-2-yl-cyclohexa-1,3-dienecarbaldehyde
44 5-methyl-6-phenyl-4-(thiophen-2-yl)cyclohexa-1,3-dienecarbaldehyde
45 4-Hexyl-6-methyl-cyclohexa-1,3-dienecarbaldehyde

### Example 10

### Anti-bacterial and Anti-fungal Activity of Certain Compounds of the Present Invention

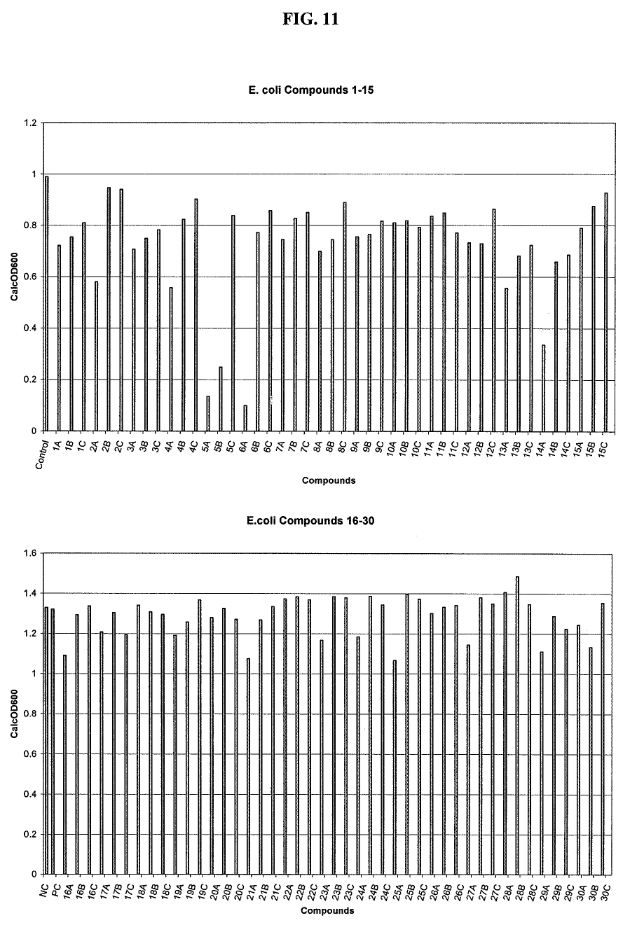

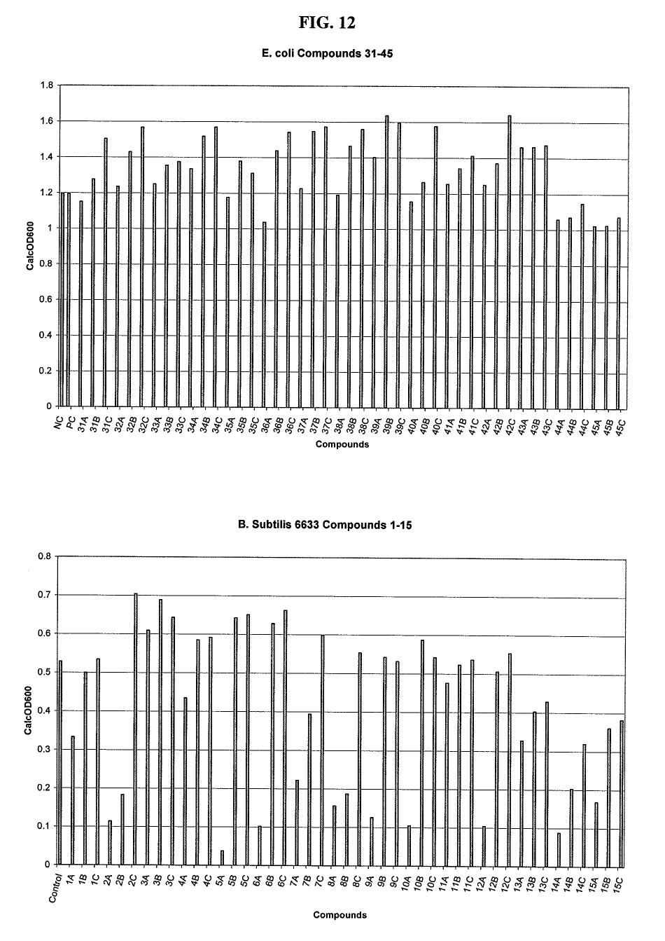

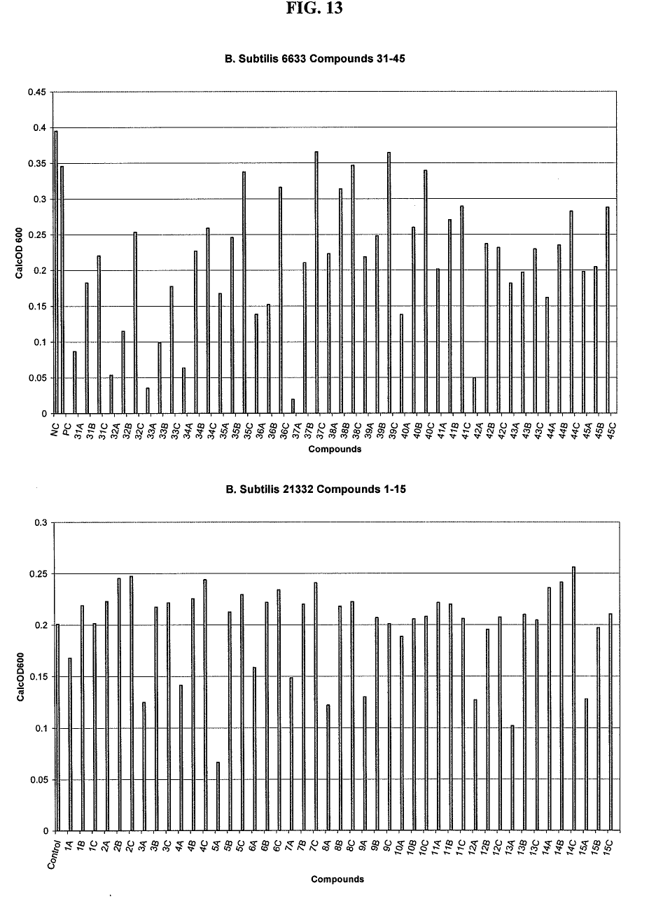

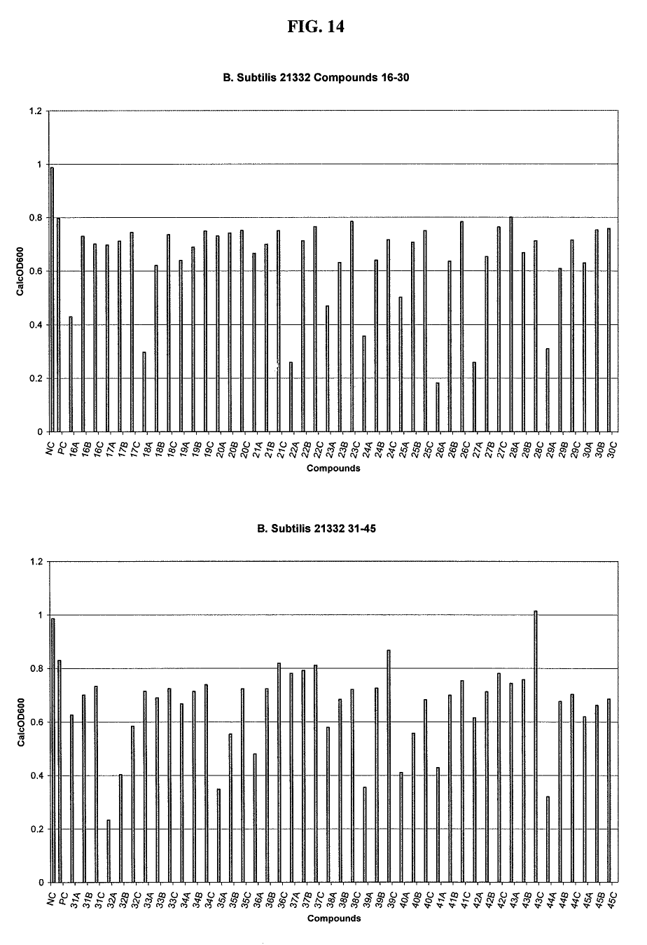

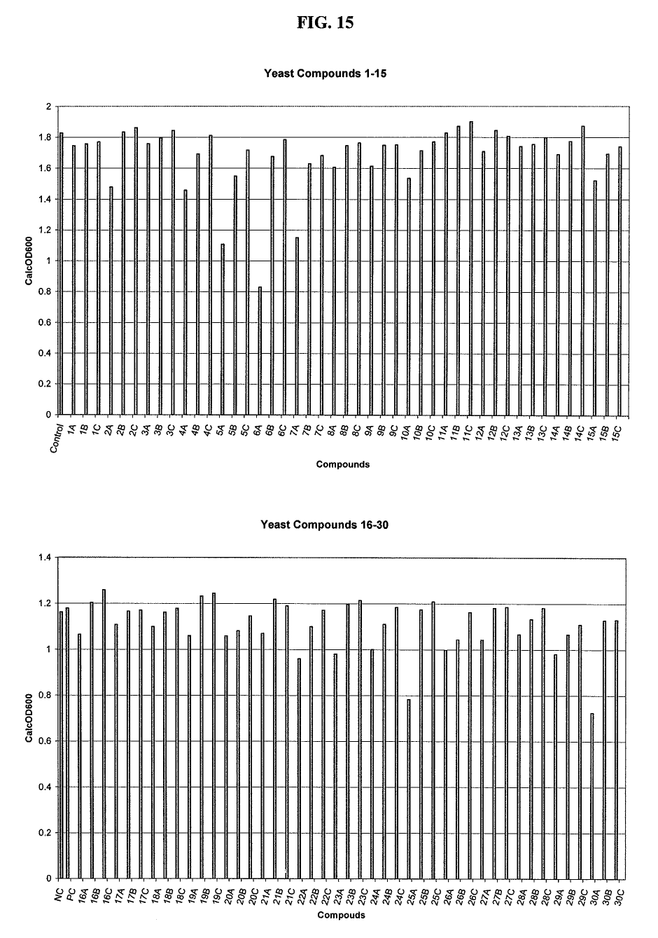

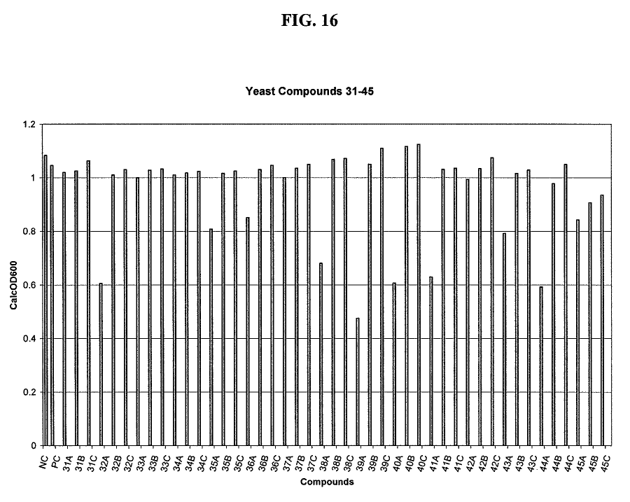

Anti-bacterial and -fungal assays were preformed as follows. Microbes (Saccharomyces cerevisiae, Escherichia coli, Bacillus subtilis 6633, Bacillus subtilis 21332, and Pseudomonas aeroginosa) were cultured and maintained on agar plates. A single colony was used to inoculate 3 mL of culture media and allowed to grow for 16 h. The cells (500 μL) were diluted with 50 mL of fresh media to an O.D. of 0.1 and aliquoted into a 96-well plate (100 μL/well). The assays were set up in duplicate and the compounds tested to give final concentrations of 100, 10 and 1 μg/mL. Plates were incubated and shaken for 16 h and cell density (O.D.) measured with a Bio-Tek microplate reader. See FIGS. 11-16 (compound numbering is the same as listed for Example 9).

All of the methods disclosed and claimed herein can be made and executed without undue experimentation in light of the present disclosure. While the methods of this invention have been described in terms of preferred embodiments, it will be apparent to those of skill in the art that variations may be applied to the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Prov. Appln. 60/786,542
Alcaide and Almendros, Angew. Chem. Int. Ed., 42:858-860, 2003.
Asato et al., Tetrahedron Lett., 33:3105-3108, 1992.
Bench et al., J. Org. Chem., 71:9458-9463, 2006.
Betancort et al., Synthesis, 9:1509-1521, 2004.
Bøgevig et al., Chem. Commun., 6:620-621, 2002.
Bundgaard, In: Design of Prodrugs, Elsevier Science, Amsterdam, 1985.
Casas et al., Angew. Chem. Int. Ed., 44:1343-1345, 2005.
Christoffers and Baro, Angew. Chem. Int. Ed., 42:1688-1690, 2003.
Córdova et al., J. Org. Chem., 67:301-303, 2002.
Dalko and Moisan, Angew. Chem. Int. Ed., 43:5138-5175, 2004.
Dalko and Moisan, Angew. Chem. Int. Ed., 43:5138-5175, 2004.
DE 2102623
DE 2439294
DE 2456958
Duhamel et al., Tetrahedron Lett., 35:4495-4498, 1991.
Eberback and Roser, Tetrahedron Lett., 28:2685-2688, 1987.
Eder et al., Chem. Int. Ed. Engl., 10:496-497, 1971.
Fischer and Löwenberg, Liebigs Ann. Chem., 494:263, 1932.
Fishkin et al., Invest. Ophthalmol. Vis. Sci., 45E: 1803, 2004.
Fishkin et al., Proc. Natl. Acad. Sci. USA, 102:7091-7096, 2005.
Green et al., In: Culturing Nerve Cells, 2^{nd} Ed., MIT Press, MA, 161-188, 1998.
Halland et al., Angew. Chem. Int. Ed., 42:4955-4957, 2003.
Halland et al., J. Org. Chem., 67:8331-8338, 2002.
Harada and Nakanishi, Acc. Chem. Res., 5:257-263, 1972.
Hechavarria Fonseca and List, Angew. Chem. Int. Ed., 43:3958-3960, 2004.
Holst et al., Acta Chem. Scand., 48:765-768, 1994.
Horstmann et al., Angew. Chem. Int. Ed., 39:3635-3638, 2000.
Hu et al., J. Org. Chem., 69:3782, 2004.
Huang et al., Huaxue Xuebao, 41:269-73, 1983.
Ishihara and Nakano, Am. Chem. Soc., 127:10504-10505, 2005.
Krause and Hoffmann-Röder, Synthesis, 171-196, 2001.
Krinsky, Pure Appl. Chem., 1994, 66:1001-1010, 1994.
Kyba et al., J. Am. Chem. Soc., 100:4555-4568, 1978.
Labbé, Bull. Soc. Chim. France, 21:407, 1899.
Lakne and Negrete, Syn. Lett., 4:2002, 2002
List et al., J. Org. Lett., 3:2423-2425, 2002.
List et al., Proc. Natl. Acad. Sci. USA, 5839-5842, 2004.
List, Acc. Chem. Res., 37:548-557, 2004.
List, Tetrahedron, 58:5573-5590, 2002.
Martini, J. Neurocytol. 23:1-28 (1994).
McFarland et al., J. Med. Chem., 12:1066-1079, 1969.
Meyers et al., J. Org. Chem., 38:36-56, 1973.
Northrup and MacMillan, J. Am. Chem. Soc., 124:6798-6799, 2002.
Northrup et al., Angew. Chem. Int. Ed., 43:2152-2154, 2004.
Notz et al., Acc. Chem. Res., 37:580-591, 2004.
Ono et al., J. Org. Chem., 47:5017-5019, 1982.
Peters et al., Appl. Environ. Microbiol., 69:3469-3475, 2003.
Peters et al., Planta Med., 70:883-886, 2004.
Rabiller and Danho, Helv. Chem. Acta, 67:1254-1273, 1984.
Ramachary et al., Tetrahedron Lett., 43:6743-6746, 2002.
Ramamurthy et al., Tetrahedron, 31:193-199, 1975.
Remington's Pharmaceutical Sciences, 18^{th} Ed., Mack Printing Co., 1289-1329, 1990.
Sabitha et al., Adv. Synth. Catal., 347:1353-1355, 2005.
Sakthivel et al., J. Am. Chem. Soc., 123:5260-5267, 2001.
Seayad and List, Org. Biomol. Chem., 3:719-724, 2005.
Sundén et al., Angew. Chem. Int. Ed., 44:4877-4880, 2005.
Syper, Tetrahedron, 43:2853-2871, 1987.
Taber et al., J. Org. Chem., 52:28-34, 1987.
Taneja et al., Indian J Chem., 27B:769-770, 1988.
Thayumanavan et al., Tetrahedron Lett., 43:3817-3820, 2002.
Thomas and Guntz-Dubin, Helv. Chim. Acta., 59:2261-2267, 1976.
Tiemann, Ber. Deutsch. Chem. Ges., 31:3278, 1898.
Tucker et al., Tetrahedron Lett., 22:1373-1376, 1981.
Uchikawa et al., J. Med. Chem., 45:4222-4239, 2002.
Valla et al., Tetrahedron Lett., 41:3359-3362, 2000.
Verdegem et al., Tetrahedron Lett., 38:5355-5358, 1997.
Yamad et al., Tetrahedron Lett., 5:377-380, 1973a.
Yamad et al., Tetrahedron Lett., 5:381-384, 1973b.
Zhou et al., Synthesis, 2:217-220, 2004.

What is claimed is:

1. A method of preparing a cyclohexadienal comprising reacting an α,β-unsaturated aldehyde with a β-substituted α,β-unsaturated aldehyde in the presence of a compound of formula (I): 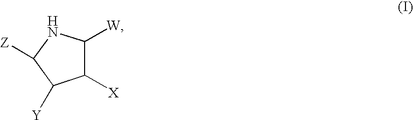
wherein W, X, Y, and Z are each independently selected from the group consisting of:
H;
halogen;
—COOR_{A}, wherein R_{A} is selected from the group consisting of H, alkyl, cycloalkyl and aryl;
—CR_{B}R_{C}R_{D}, wherein R_{B}, R_{C} and R_{D} are each independently selected from the group consisting of H, alkyl, cycloalkyl, aryl, —CN and halogen,
or wherein R_{B}, R_{C}, and/or R_{D} taken together are comprised in a cyclic moiety with C,
or wherein R_{C} is not present;
—C═N—R_{E}, wherein R_{E} is selected from the group consisting of alkyl, cycloalkyl and aryl;
—NR_{F}R_{G}, wherein R_{F} and R_{G} are independently selected from the group consisting of H, alkyl, cycloalkyl and aryl,
or wherein R_{F} and R_{G} taken together are comprised in a cyclic moiety with N;
—N═R_{H}, wherein R_{H} is selected from the group consisting of alkyl, cycloalkyl and aryl; and
—OR_{I}, wherein R_{I} is selected from the group consisting of alkyl, cycloalkyl and aryl;
or wherein W and X, X and Y, and/or Y and Z taken together are comprised in a cyclic moiety;
provided that the compound of formula (I) is not D-proline.

2. The method of claim 1, wherein the α,β-unsaturated aldehyde is of formula (II): 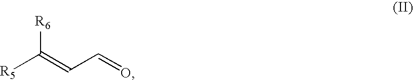
wherein R₅ and R₆ are each independently selected from the group consisting of:
H;
halogen;
—CR_{A}R_{B}R_{C}, wherein R_{A}, R_{B} and R_{C} are each independently selected from the group consisting of H, alkyl, cycloalkyl, aryl, —CN and halogen,
or wherein R_{A}, R_{B}, and/or R_{C} taken together are comprised in a cyclic moiety with C,
or wherein R_{C} is not present;
—C═N—R_{D}, wherein R_{D} is selected from the group consisting of alkyl, cycloalkyl and aryl;
—NR_{E}R_{F}, wherein R_{E} and R_{F} are independently selected from the group consisting of H, alkyl, cycloalkyl and aryl,
or wherein R_{E} and R_{F} taken together are comprised in a cyclic moiety with N;
—N═R_{G}, wherein R_{G} is selected from the group consisting of alkyl, cycloalkyl and aryl; and
—OR_{H}, wherein R_{H} is selected from the group consisting of alkyl, cycloalkyl and aryl;
or wherein R₅ and R₆ taken together are comprised in a cyclic moiety.

3. The method of claim 1, wherein the β-substituted α,β-unsaturated aldehyde is of formula (III): 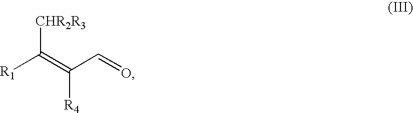
wherein R₁, R₂, R₃, and R₄ are each independently selected from the group consisting of:
—H;
halogen;
—CR_{A}R_{B}R_{C}, wherein R_{A}, R_{B} and R_{C} are each independently selected from the group consisting of H, alkyl, cycloalkyl, aryl, —CN and halogen,
or wherein R_{A}, R_{B}, and/or R_{C} taken together are comprised in a cyclic moiety with C
or wherein R_{C} is not present;
—C═N—R_{D}, wherein R_{D} is selected from the group consisting of alkyl, cycloalkyl and aryl;
—NR_{E}R_{F}, wherein R_{E} and R_{F} are independently selected from the group consisting of H, alkyl, cycloalkyl and aryl,
or wherein R_{E} and R_{F} taken together are comprised in a cyclic moiety with N;
—N═R_{G}, wherein R_{G} is selected from the group consisting of alkyl, cycloalkyl and aryl; and
—OR_{H}, wherein R_{H} is selected from the group consisting of alkyl, cycloalkyl and aryl,
or wherein R₁ and R₂, R₁ and R₄, and/or R₂ and R₃ taken together are comprised in a cyclic moiety.

4. The method of claim 1, wherein W is COOR_{A}.

5. The method of claim 4, wherein R_{A} is H.

6. The method of claim 4, wherein R_{A} is an alkyl group.

7. The method of claim 6, wherein the alkyl group is selected from the group consisting of methyl, isopropyl and benzyl.

8. The method of claim 1, wherein W is alkyl.

9. The method of claim 8, wherein the alkyl group is selected from the group consisting of —CH₂OH, —C(H)(OH)((CH₂)(CH₃))₂ and —C(H)(OH)(phenyl)₂.

10. The method of claim 1, wherein X, Y and Z are each H.

11. The method of claim 1, wherein the compound of formula (I) is further defined as L-proline.

12. The method of claim 1, wherein the reaction method takes place in the presence of ethanol.

13. The method of claim 1, wherein the α,β-unsaturated aldehyde is the same compound as the β-substituted α,β-unsaturated aldehyde.

14. The method of claim 13, wherein the molar ratio of the α,β-unsaturated aldehyde to the β-substituted α,β-unsaturated aldehyde is about 1:1.5.

15. The method of claim 1, wherein the α,β-unsaturated aldehyde is a different compound than the β-substituted α,β-unsaturated aldehyde.

16. The method of claim 1, wherein R₁, R₂, R₃, R₄, R₅ and R₆ are each independently selected from the group consisting of H, alkyl, cycloalkyl and aryl.

17. The method of claim 1, wherein only one of R₁, R₂, R₃, R₄, R₅ and R₆ is H.

18. The method of claim 17, wherein only one of R₂ and R₃ is H.

19. The method of claim 1, wherein the cyclohexadienal is substantially pure.

20. The method of claim 1, wherein the cyclohexadienal is selected from the group consisting of 6-methyl-4,6-bis-(4-methyl-pent-3-enyl)-cyclohexa-1,3-dienecarbaldehyde; 6,6-dimethyl-4-(4-methyl-pent-3-enyl)-cyclohexa-1,3-dienecarbaldehyde; 6-methyl-4-(4-methyl-pent-3-enyl)-cyclohexa-1,3-dienecarbaldehyde; 4-(4-methyl-pent-3-enyl)-6-phenyl-cyclohexa-1,3-dienecarbaldehyde; 4-(4-methyl-pent-3-enyl)-6-(4-nitro-phenyl)-cyclohexa-1,3-dienecarbaldehyde; 6-(4-dimethylamino-phenyl)-4-(4-methyl-pent-3-enyl)-cyclohexa-1,3-dienecarbaldehyde; 4-(4-methyl-pent-3-enyl)-cyclohexa-1,3-dienecarbaldehyde; 6-methyl-4,6-bis-(5-methyl-furan-2-yl)-cyclohexa-1,3-dienecarbaldehyde; 4-(5-methyl-furan-2-yl)-6-phenyl-cyclohexa-1,3-dienecarbaldehyde; 4-(5-methyl-furan-2-yl)-6-(4-nitro-phenyl)-cyclohexa-1,3-dienecarbaldehyde; 6-(4-dimethylamino-phenyl)-4-(5-methyl-furan-2-yl)-cyclohexa-1,3-dienecarbaldehyde; 6-methyl-4,6-di-thiophen-2-yl-cyclohexa-1,3-dienecarbaldehyde; 6-(4-dimethylamino-phenyl)-4-thiophen-2-yl-cyclohexa-1,3-dienecarbaldehyde; 6-(4-nitro-phenyl)-4-thiophen-2-yl-cyclohexa-1,3-dienecarbaldehyde; 6-phenyl-4-thiophen-2-yl-cyclohexa-1,3-dienecarbaldehyde; 6-methyl-4-thiophen-2-yl-cyclohexa-1,3-dienecarbaldehyde; 6-(4-dimethylamino-phenyl)-4-(1H-indol-3-yl)-cyclohexa-1,3-dienecarbaldehyde; 4,6-bis-(1H-indol-3-yl)-6-methyl-cyclohexa-1,3-dienecarbaldehyde; 4-methyl-6-phenyl-cyclohexa-1,3-dienecarbaldehyde; 4-hept-1-enyl-6-methyl-cyclohexa-1,3-dienecarbaldehyde; 4,6-di-hept-1-enyl-6-methyl-cyclohexa-1,3-dienecarbaldehyde; 4,6-bis-biphenyl-4-yl-6-methyl-cyclohexa-1,3-dienecarbaldehyde; 4-biphenyl-4-yl-6-methyl-cyclohexa-1,3-dienecarbaldehyde; 4-biphenyl-4-yl-6-phenyl-cyclohexa-1,3-dienecarbaldehyde; 4-biphenyl-4-yl-6-(4-nitro-phenyl)-cyclohexa-1,3-dienecarbaldehyde; 4-biphenyl-4-yl-6-(4-dimethylamino-phenyl)-cyclohexa-1,3-dienecarbaldehyde; 4,6-bis-(9H-fluoren-2-yl)-6-methyl-cyclohexa-1,3-dienecarbaldehyde; 4-(9H-fluoren-2-yl)-6-methyl-cyclohexa-1,3-dienecarbaldehyde; 4-(9H-fluoren-2-yl)-6-phenyl-cyclohexa-1,3-dienecarbaldehyde; 6-(4-dimethylamino-phenyl)-4-(9H-fluoren-2-yl)-cyclohexa-1,3-dienecarbaldehyde; 4-(9H-fluoren-2-yl)-6-(4-nitro-phenyl)-cyclohexa-1,3-dienecarbaldehyde; 6-methyl-4-[2-(2,6,6-trimethyl-cyclohex-1-enyl)-vinyl]-cyclohexa-1,3-dienecarbaldehyde; 6-phenyl-4-[2-(2,6,6-trimethyl-cyclohex-1-enyl)-vinyl]-cyclohexa-1,3-dienecarbaldehyde; 6-(4-nitro-phenyl)-4-[2-(2,6,6-trimethyl-cyclohex-1-enyl)-vinyl]-cyclohexa-1,3-dienecarbaldehyde; 6-(4-dimethylamino-phenyl)-4-[2-(2,6,6-trimethyl-cyclohex-1-enyl)-vinyl]-cyclohexa-1,3-dienecarbaldehyde; 6-methyl-4,6-bis-[2-(2,6,6-trimethyl-cyclohex-1-enyl)-vinyl]-cyclohexa-1,3-dienecarbaldehyde; 6-methyl-6-[3-methyl-6-(2,6,6-trimethyl-cyclohex-1-enyl)-hexa-1,3,5-trienyl]-4-[4-methyl-6-(2,6,6-trimethyl-cyclohex-1-enyl)-hexa-1,3,5-trienyl]-cyclohexa-1,3-dienecarbaldehyde; 6-methyl-4-[4-methyl-6-(2,6,6-trimethyl-cyclohex-1-enyl)-hexa-1,3,5-trienyl]-cyclohexa-1,3-dienecarbaldehyde; 4-[4-methyl-6-(2,6,6-trimethyl-cyclohex-1-enyl)-hexa-1,3,5-trienyl]-6-phenyl-cyclohexa-1,3-dienecarbaldehyde; 6-(4-dimethylamino-phenyl)-4-[4-methyl-6-(2,6,6-trimethyl-cyclohex-1-enyl)-hexa-1,3,5-trienyl]-cyclohexa-1,3-dienecarbaldehyde; 4-[4-methyl-6-(2,6,6-trimethyl-cyclohex-1-enyl)-hexa-1,3,5-trienyl]-6-(4-nitro-phenyl)-cyclohexa-1,3-dienecarbaldehyde; 6-methyl-4-(4-methyl-pent-3-enyl)-6-thiophen-2-yl-cyclohexa-1,3-dienecarbaldehyde; 4-biphenyl-4-yl-6-methyl-6-(5-methyl-furan-2-yl)-cyclohexa-1,3-dienecarbaldehyde; 4,6-dihexyl-6-methyl-cyclohexa-1,3-dienecarbaldehyde; 4-hexyl-6-methyl-cyclohexa-1,3-dienecarbaldehyde; 4-biphenyl-4-yl-6-methyl-6-thiophen-2-yl-cyclohexa-1,3-dienecarbaldehyde; (E)-6,6-dimethyl-4-(2-(2,6,6-trimethylcyclohex-1-enyl)vinyl)cyclohexa-1,3-dienecarbaldehyde; 6-methyl-6-(5-methylfuran-2-yl)-4-(4-methylpent-3-enyl)cyclohexa-1,3-dienecarbaldehyde; 6,6-dimethyl-4-(5-methylfuran-2-yl)cyclohexa-1,3-dienecarbaldehyde; 6-methyl-4-(naphthalen-2-yl)cyclohexa-1,3-dienecarbaldehyde; (E)-4-(4,8-dimethylnona-3,7-dienyl)-6-methylcyclohexa-1,3-dienecarbaldehyde; 5,6-dimethyl-4-(thiophen-2-yl)cyclohexa-1,3-dienecarbaldehyde; 5,6,6-trimethyl-4-(thiophen-2-yl)cyclohexa-1,3-dienecarbaldehyde; 6-(biphenyl-4-yl)-4,6-dimethylcyclohexa-1,3-dienecarbaldehyde; 6,6-dimethyl-4-(naphthalen-2-yl)cyclohexa-1,3-dienecarbaldehyde; 4-(biphenyl-4-yl)-6,6-dimethylcyclohexa-1,3-dienecarbaldehyde; 4,6,6-trimethylcyclohexa-1,3-dienecarbaldehyde; 5,6-dimethyl-4,6-di(thiophen-2-yl)cyclohexa-1,3-dienecarbaldehyde; 6-methyl-4,6-diphenylcyclohexa-1,3-dienecarbaldehyde; 6-methyl-4,6-di(naphthalen-2-yl)cyclohexa-1,3-dienecarbaldehyde; 4,6-bis((E)-4,8-dimethylnona-3,7-dienyl)-6-methylcyclohexa-1,3-dienecarbaldehyde; cis-4-methyl-5-(3-methyl-but-2-enyl)-6-phenyl-cyclohexa-1,3-dienecarbaldehyde; trans-4-methyl-5-(3-methyl-but-2-enyl)-6-phenyl-cyclohexa-1,3-dienecarbaldehyde; cis and trans-6-(4-dimethylamino-phenyl)-4-methyl-5-(3-methyl-but-2-enyl)-cyclohexa-1,3-dienecarbaldehyde; 6-(4-(dimethylamino)phenyl)-4-methylcyclohexa-1,3-dienecarbaldehyde; 4-methyl-6-(4-nitrophenyl)cyclohexa-1,3-dienecarbaldehyde; 4-methylcyclohexa-1,3-dienecarbaldehyde; 4-hexyl-6-phenylcyclohexa-1,3-dienecarbaldehyde; 6-(4-(dimethylamino)phenyl)-4-hexylcyclohexa-1,3-dienecarbaldehyde; 4,6-diphenylcyclohexa-1,3-dienecarbaldehyde; 6-(4-(dimethylamino)phenyl)-4-phenylcyclohexa-1,3-dienecarbaldehyde; 6-(4-nitrophenyl)-4-phenylcyclohexa-1,3-dienecarbaldehyde; 4-(naphthalen-2-yl)-6-phenylcyclohexa-1,3-dienecarbaldehyde; 6-(4-(dimethylamino)phenyl)-4-(naphthalen-2-yl)cyclohexa-1,3-dienecarbaldehyde; 4-(naphthalen-2-yl)-6-(4-nitrophenyl)cyclohexa-1,3-dienecarbaldehyde; 4-(naphthalen-2-yl)cyclohexa-1,3-dienecarbaldehyde; (E)-4-(4,8-dimethylnona-3,7-dienyl)cyclohexa-1,3-dienecarbaldehyde; (E)-4-(4,8-dimethylnona-3,7-dienyl)-6-phenylcyclohexa-1,3-dienecarbaldehyde; (E)-6-(4-(dimethylamino)phenyl)-4-(4,8-dimethylnona-3,7-dienyl)cyclohexa-1,3-dienecarbaldehyde; 5-methyl-6-(4-nitrophenyl)-4-(thiophen-2-yl)cyclohexa-1,3-dienecarbaldehyde; 6-(4-(dimethylamino)phenyl)-5-methyl-4-(thiophen-2-yl)cyclohexa-1,3-dienecarbaldehyde; 5-methyl-6-phenyl-4-(thiophen-2-yl)cyclohexa-1,3-dienecarbaldehyde; 5-methyl-4-(thiophen-2-yl)cyclohexa-1,3-dienecarbaldehyde; 5-(4-methyl-pent-3-enyl)-4′-nitro-biphenyl-2-carbaldehyde; 4″-dimethylamino-[1,1′;3′,1″]terphenyl-4′-carbaldehyde; 4′″-dimethylamino-[1,1′;4′,1″;3″,1′″]quaterphenyl-4″-carbaldehyde; 4′-dimethylamino-5-(9H-fluoren-2-yl)-biphenyl-2-carbaldehyde; 4′-(dimethylamino)-5-methylbiphenyl-2-carbaldehyde; 5-(9H-fluoren-2-yl)-biphenyl-2-carbaldehyde; and 5-(4-methyl-pent-3-enyl)-biphenyl-2-carbaldehyde.

21. The method of claim 1, wherein the cyclohexadienal is further comprised in a pharmaceutical composition.

22. A method of stimulating neurite growth, comprising administering to a cell an effective amount of a cyclohexadienal of claim 1.

23. The method of claim 22, wherein the cyclohexadienal comprises a substituent selected from the group consisting of naphthyl, biphenyl and farnesyl.

24. The method of claim 22, wherein the cyclohexadienal of claim 1 is further defined as 4-(biphenyl-4-yl)-6-methyl-6-(5-methylfuran-2-yl)cyclohexa-1,3-dienecarbaldehyde.

25. The method of claim 22, wherein the cyclohexadienal of claim 1 is further defined as 4,6-dihexyl-6-methylcyclohexa-1,3-dienecarbaldehyde.

26. The method of claim 22, wherein the cyclohexadienal of claim 1 is further defined as 6-(4-(dimethylamino)phenyl)-4-(naphthalen-2-yl)cyclohexa-1,3-dienecarbaldehyde.

27. In an improved method for reacting an α,β-unsaturated aldehyde with a β-substituted α,β-unsaturated aldehyde, the improvement comprising performing the reaction in the presence of a compound of formula (I): 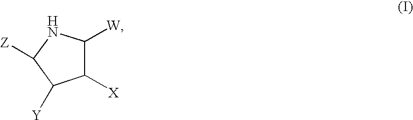
wherein W, X, Y, and Z are each independently selected from the group consisting of:
H;
halogen;
—COOR_{A}, wherein R_{A} is selected from the group consisting of H, alkyl, cycloalkyl and aryl;
—CR_{B}R_{C}R_{D}, wherein R_{B}, R_{C} and R_{D} are each independently selected from the group consisting of H, alkyl, cycloalkyl, aryl, —CN and halogen,
or wherein R_{B}, R_{C}, and/or R_{D} taken together are comprised in a cyclic moiety with C,
or wherein R_{C} is not present;
—C═N—R_{E}, wherein R_{E} is selected from the group consisting of alkyl, cycloalkyl and aryl;
—NR_{F}R_{G}, wherein R_{F} and R_{G} are independently selected from the group consisting of H, alkyl, cycloalkyl and aryl,
or wherein R_{F} and R_{G} taken together are comprised in a cyclic moiety with N;
—N═R_{H}, wherein R_{H} is selected from the group consisting of alkyl, cycloalkyl and aryl; and
—OR_{I}, wherein R_{I} is selected from the group consisting of alkyl, cycloalkyl and aryl;
or wherein W and X, X and Y, and/or Y and Z taken together are comprised in a cyclic moiety;
provided that the compound of formula (I) is not D-proline.

28. A cyclohexadienal of formula (IV): 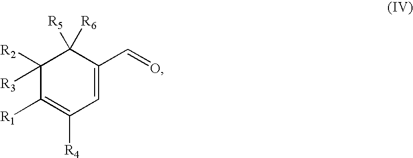
wherein R₁, R₂, R₃, R₄, R₅ and R₆ are each independently selected from the group consisting of:
H;
halogen;
—CR_{A}R_{B}R_{C}, wherein R_{A}, R_{B} and R_{C} are each independently selected from the group consisting of H, alkyl, cycloalkyl, aryl, —CN and halogen,
or wherein R_{A}, R_{B}, and/or R_{C} taken together are comprised in a cyclic moiety with C,
or wherein R_{C} is not present;
—C═N—R_{D}, wherein R_{D} is selected from the group consisting of alkyl, cycloalkyl and aryl;
—NR_{E}R_{F}, wherein R_{E} and R_{F} are independently selected from the group consisting of H, alkyl, cycloalkyl and aryl,
or wherein R_{E} and R_{F} taken together are comprised in a cyclic moiety with N;
—N═R_{G}, wherein R_{G} is selected from the group consisting of alkyl, cycloalkyl and aryl; and
—OR_{H}, wherein R_{H} is selected from the group consisting of alkyl, cycloalkyl and aryl;
or wherein R₁ and R₂, R₁ and R₄, R₂ and R₃, and/or R₅ and R₆ taken together form a cyclic moiety;
provided that the compound of formula (IV) is neither A nor B:

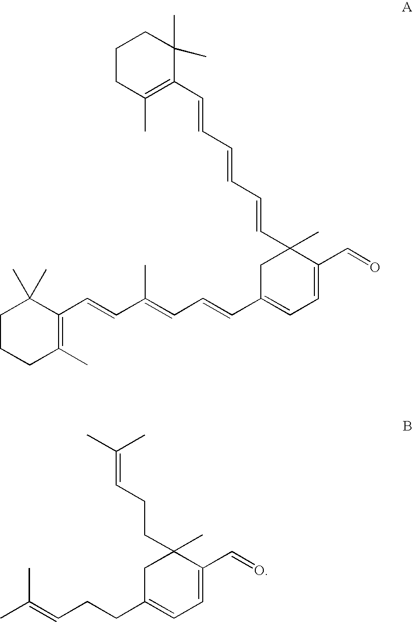

29. The cyclohexadienal of claim 28, wherein R₁ is selected from the group consisting of alkyl, cycloalkyl and aryl.

30. The cyclohexadienal of claim 28, wherein R₅ and R₆ are independently selected from the group consisting of H, alkyl, cycloalkyl and aryl.

31. The cyclohexadienal of claim 30, wherein either R₅ or R₆ is an aryl group.

32. The cyclohexadienal of claim 31, wherein the aryl group is a phenyl group.

33. The cyclohexadienal of claim 28, wherein the cyclohexadienal is selected from the group consisting of 6-methyl-4,6-bis-(4-methyl-pent-3-enyl)-cyclohexa-1,3-dienecarbaldehyde; 6,6-dimethyl-4-(4-methyl-pent-3-enyl)-cyclohexa-1,3-dienecarbaldehyde; 6-methyl-4-(4-methyl-pent-3-enyl)-cyclohexa-1,3-dienecarbaldehyde; 4-(4-methyl-pent-3-enyl)-6-phenyl-cyclohexa-1,3-dienecarbaldehyde; 4-(4-methyl-pent-3-enyl)-6-(4-nitro-phenyl)-cyclohexa-1,3-dienecarbaldehyde; 6-(4-dimethylamino-phenyl)-4-(4-methyl-pent-3-enyl)-cyclohexa-1,3-dienecarbaldehyde; 4-(4-methyl-pent-3-enyl)-cyclohexa-1,3-dienecarbaldehyde; 6-methyl-4,6-bis-(5-methyl-furan-2-yl)-cyclohexa-1,3-dienecarbaldehyde; 4-(5-methyl-furan-2-yl)-6-phenyl-cyclohexa-1,3-dienecarbaldehyde; 4-(5-methyl-furan-2-yl)-6-(4-nitro-phenyl)-cyclohexa-1,3-dienecarbaldehyde; 6-(4-dimethylamino-phenyl)-4-(5-methyl-furan-2-yl)-cyclohexa-1,3-dienecarbaldehyde; 6-methyl-4,6-di-thiophen-2-yl-cyclohexa-1,3-dienecarbaldehyde; 6-(4-dimethylamino-phenyl)-4-thiophen-2-yl-cyclohexa-1,3-dienecarbaldehyde; 6-(4-nitro-phenyl)-4-thiophen-2-yl-cyclohexa-1,3-dienecarbaldehyde; 6-phenyl-4-thiophen-2-yl-cyclohexa-1,3-dienecarbaldehyde; 6-methyl-4-thiophen-2-yl-cyclohexa-1,3-dienecarbaldehyde; 6-(4-dimethylamino-phenyl)-4-(1H-indol-3-yl)-cyclohexa-1,3-dienecarbaldehyde; 4,6-bis-(1H-indol-3-yl)-6-methyl-cyclohexa-1,3-dienecarbaldehyde; 4-methyl-6-phenyl-cyclohexa-1,3-dienecarbaldehyde; 4-hept-1-enyl-6-methyl-cyclohexa-1,3-dienecarbaldehyde; 4,6-di-hept-1-enyl-6-methyl-cyclohexa-1,3-dienecarbaldehyde; 4,6-bis-biphenyl-4-yl-6-methyl-cyclohexa-1,3-dienecarbaldehyde; 4-biphenyl-4-yl-6-methyl-cyclohexa-1,3-dienecarbaldehyde; 4-biphenyl-4-yl-6-phenyl-cyclohexa-1,3-dienecarbaldehyde; 4-biphenyl-4-yl-6-(4-nitro-phenyl)-cyclohexa-1,3-dienecarbaldehyde; 4-biphenyl-4-yl-6-(4-dimethylamino-phenyl)-cyclohexa-1,3-dienecarbaldehyde; 4,6-bis-(9H-fluoren-2-yl)-6-methyl-cyclohexa-1,3-dienecarbaldehyde; 4-(9H-fluoren-2-yl)-6-methyl-cyclohexa-1,3-dienecarbaldehyde; 4-(9H-fluoren-2-yl)-6-phenyl-cyclohexa-1,3-dienecarbaldehyde; 6-(4-dimethylamino-phenyl)-4-(9H-fluoren-2-yl)-cyclohexa-1,3-dienecarbaldehyde; 4-(9H-fluoren-2-yl)-6-(4-nitro-phenyl)-cyclohexa-1,3-dienecarbaldehyde; 6-methyl-4-[2-(2,6,6-trimethyl-cyclohex-1-enyl)-vinyl]-cyclohexa-1,3-dienecarbaldehyde; 6-phenyl-4-[2-(2,6,6-trimethyl-cyclohex-1-enyl)-vinyl]-cyclohexa-1,3-dienecarbaldehyde; 6-(4-nitro-phenyl)-4-[2-(2,6,6-trimethyl-cyclohex-1-enyl)-vinyl]-cyclohexa-1,3-dienecarbaldehyde; 6-(4-dimethylamino-phenyl)-4-[2-(2,6,6-trimethyl-cyclohex-1-enyl)-vinyl]-cyclohexa-1,3-dienecarbaldehyde; 6-methyl-4,6-bis-[2-(2,6,6-trimethyl-cyclohex-1-enyl)-vinyl]-cyclohexa-1,3-dienecarbaldehyde; 6-methyl-6-[3-methyl-6-(2,6,6-trimethyl-cyclohex-1-enyl)-hexa-1,3,5-trienyl]-4-[4-methyl-6-(2,6,6-trimethyl-cyclohex-1-enyl)-hexa-1,3,5-trienyl]-cyclohexa-1,3-dienecarbaldehyde; 6-methyl-4-[4-methyl-6-(2,6,6-trimethyl-cyclohex-1-enyl)-hexa-1,3,5-trienyl]-cyclohexa-1,3-dienecarbaldehyde; 4-[4-methyl-6-(2,6,6-trimethyl-cyclohex-1-enyl)-hexa-1,3,5-trienyl]-6-phenyl-cyclohexa-1,3-dienecarbaldehyde; 6-(4-dimethylamino-phenyl)-4-[4-methyl-6-(2,6,6-trimethyl-cyclohex-1-enyl)-hexa-1,3,5-trienyl]-cyclohexa-1,3-dienecarbaldehyde; 4-[4-methyl-6-(2,6,6-trimethyl-cyclohex-1-enyl)-hexa-1,3,5-trienyl]-6-(4-nitro-phenyl)-cyclohexa-1,3-dienecarbaldehyde; 6-methyl-4-(4-methyl-pent-3-enyl)-6-thiophen-2-yl-cyclohexa-1,3-dienecarbaldehyde; 4-biphenyl-4-yl-6-methyl-6-(5-methyl-furan-2-yl)-cyclohexa-1,3-dienecarbaldehyde; 4,6-dihexyl-6-methyl-cyclohexa-1,3-dienecarbaldehyde; 4-hexyl-6-methyl-cyclohexa-1,3-dienecarbaldehyde; 4-biphenyl-4-yl-6-methyl-6-thiophen-2-yl-cyclohexa-1,3-dienecarbaldehyde; (E)-6,6-dimethyl-4-(2-(2,6,6-trimethylcyclohex-1-enyl)vinyl)cyclohexa-1,3-dienecarbaldehyde; 6-methyl-6-(5-methylfuran-2-yl)-4-(4-methylpent-3-enyl)cyclohexa-1,3-dienecarbaldehyde; 6,6-dimethyl-4-(5-methylfuran-2-yl)cyclohexa-1,3-dienecarbaldehyde; 6-methyl-4-(naphthalen-2-yl)cyclohexa-1,3-dienecarbaldehyde; (E)-4-(4,8-dimethylnona-3,7-dienyl)-6-methylcyclohexa-1,3-dienecarbaldehyde; 5,6-dimethyl-4-(thiophen-2-yl)cyclohexa-1,3-dienecarbaldehyde; 5,6,6-trimethyl-4-(thiophen-2-yl)cyclohexa-1,3-dienecarbaldehyde; 6-(biphenyl-4-yl)-4,6-dimethylcyclohexa-1,3-dienecarbaldehyde; 6,6-dimethyl-4-(naphthalen-2-yl)cyclohexa-1,3-dienecarbaldehyde; 4-(biphenyl-4-yl)-6,6-dimethylcyclohexa-1,3-dienecarbaldehyde; 4,6,6-trimethylcyclohexa-1,3-dienecarbaldehyde; 5,6-dimethyl-4,6-di(thiophen-2-yl)cyclohexa-1,3-dienecarbaldehyde; 6-methyl-4,6-diphenylcyclohexa-1,3-dienecarbaldehyde; 6-methyl-4,6-di(naphthalen-2-yl)cyclohexa-1,3-dienecarbaldehyde; 4,6-bis((E)-4,8-dimethylnona-3,7-dienyl)-6-methylcyclohexa-1,3-dienecarbaldehyde; cis-4-methyl-5-(3-methyl-but-2-enyl)-6-phenyl-cyclohexa-1,3-dienecarbaldehyde; trans-4-methyl-5-(3-methyl-but-2-enyl)-6-phenyl-cyclohexa-1,3-dienecarbaldehyde; cis and trans-6-(4-dimethylamino-phenyl)-4-methyl-5-(3-methyl-but-2-enyl)-cyclohexa-1,3-dienecarbaldehyde; 6-(4-(dimethylamino)phenyl)-4-methylcyclohexa-1,3-dienecarbaldehyde; 4-methyl-6-(4-nitrophenyl)cyclohexa-1,3-dienecarbaldehyde; 4-methylcyclohexa-1,3-dienecarbaldehyde; 4-hexyl-6-phenylcyclohexa-1,3-dienecarbaldehyde; 6-(4-(dimethylamino)phenyl)-4-hexylcyclohexa-1,3-dienecarbaldehyde; 4,6-diphenylcyclohexa-1,3-dienecarbaldehyde; 6-(4-(dimethylamino)phenyl)-4-phenylcyclohexa-1,3-dienecarbaldehyde; 6-(4-nitrophenyl)-4-phenylcyclohexa-1,3-dienecarbaldehyde; 4-(naphthalen-2-yl)-6-phenylcyclohexa-1,3-dienecarbaldehyde; 6-(4-(dimethylamino)phenyl)-4-(naphthalen-2-yl)cyclohexa-1,3-dienecarbaldehyde; 4-(naphthalen-2-yl)-6-(4-nitrophenyl)cyclohexa-1,3-dienecarbaldehyde; 4-(naphthalen-2-yl)cyclohexa-1,3-dienecarbaldehyde; (E)-4-(4,8-dimethylnona-3,7-dienyl)cyclohexa-1,3-dienecarbaldehyde; (E)-4-(4,8-dimethylnona-3,7-dienyl)-6-phenylcyclohexa-1,3-dienecarbaldehyde; (E)-6-(4-(dimethylamino)phenyl)-4-(4,8-dimethylnona-3,7-dienyl)cyclohexa-1,3-dienecarbaldehyde; 5-methyl-6-(4-nitrophenyl)-4-(thiophen-2-yl)cyclohexa-1,3-dienecarbaldehyde; 6-(4-(dimethylamino)phenyl)-5-methyl-4-(thiophen-2-yl)cyclohexa-1,3-dienecarbaldehyde; 5-methyl-6-phenyl-4-(thiophen-2-yl)cyclohexa-1,3-dienecarbaldehyde; 5-methyl-4-(thiophen-2-yl)cyclohexa-1,3-dienecarbaldehyde; 5-(4-methyl-pent-3-enyl)-4′-nitro-biphenyl-2-carbaldehyde; 4″-dimethylamino-[1,1′;3′,1″]terphenyl-4′-carbaldehyde; 4′″-dimethylamino-[1,1′;4′,1″;3″,1′″]quaterphenyl-4″-carbaldehyde; 4′-dimethylamino-5-(9H-fluoren-2-yl)-biphenyl-2-carbaldehyde; 4′-(dimethylamino)-5-methylbiphenyl-2-carbaldehyde; 5-(9H-fluoren-2-yl)-biphenyl-2-carbaldehyde; and 5-(4-methyl-pent-3-enyl)-biphenyl-2-carbaldehyde.

34. The cyclohexadienal of claim 28, further defined as 4-(biphenyl-4-yl)-6-methyl-6-(5-methylfuran-2-yl)cyclohexa-1,3-dienecarbaldehyde.

35. The cyclohexadienal of claim 28, further defined as 4,6-dihexyl-6-methylcyclohexa-1,3-dienecarbaldehyde.

36. The cyclohexadienal of claim 28, further defined as 6-(4-(dimethylamino)phenyl)-4-(naphthalen-2-yl)cyclohexa-1,3-dienecarbaldehyde.

37. The cyclohexadienal of claim 28, wherein the cyclohexadienal is substantially pure.

38. A pharmaceutical composition comprising a cyclohexadienal of claim 28.

39. A method of preparing a fluorescent compound, comprising aromatization of a cyclohexadienal of claim 28.

40. The method of claim 39, wherein the aromatization occurs naturally.

41. The method of claim 40, wherein either R₅ or R₆ of the cyclohexadienal of claim 28 is an aryl group.

42. The method of claim 41, wherein the aryl group is a phenyl group.

43. The method of claim 40, wherein the cyclohexadienal of claim 28 is further defined as 4-(4-methyl-pent-3-enyl)-6-(4-nitro-phenyl)-cyclohexa-1,3-dienecarbaldehyde.

44. The method of claim 39, wherein the aromatization comprises reacting the cyclohexadienal of claim 28 in the presence of potassium permanganate under acidic conditions.

45. The method of claim 44, wherein either R₅ or R₆ of the cyclohexadienal of claim 28 is an aryl group.

46. The method of claim 45, wherein the aryl group is a phenyl group.

47. The method of claim 44, wherein the cyclohexadienal of claim 28 is further defined as 4-(4-methylpent-3-enyl)-6-phenylcyclohexa-1,3-dienecarbaldehyde.

48. A method of stimulating neurite growth, comprising administering to a cell an effective amount of the cyclohexadienal of claim 28.

49. The method of claim 48, wherein the cyclohexadienal of claim 28 comprises a substituent selected from the group consisting of naphthyl, biphenyl and farnesyl.

50. The method of claim 49, wherein the substituent of the cyclohexadienal of claim 28 is at position R₅ or R₆.

51. The method of claim 48, wherein the cyclohexadienal is selected from the group consisting of 4-(biphenyl-4-yl)-6-methyl-6-(5-methylfuran-2-yl)cyclohexa-1,3-dienecarbaldehyde, 4,6-dihexyl-6-methylcyclohexa-1,3-dienecarbaldehyde and 6-(4-(dimethylamino)phenyl)-4-(naphthalen-2-yl)cyclohexa-1,3-dienecarbaldehyde.
